(19) United States
(12) Patent Application Publication
(10) Pub. No.: US 2024/0272152 A1
(43) Pub. Date: Aug. 15, 2024

(54) LABELING NANOSTRUCTURE FOR SIGNAL AMPLIFICATION IN IMMUNOASSAYS AND IMMUNOASSAYS USING THE LABELING NANOSTRUCTURE

(71) Applicant: Ludwig-Maximilians-Universität München, München (DE)

(72) Inventors: Tim Liedl, München (DE); Maximilian Julius Urban, München (DE); Philipp Nickels, München (DE); Linh Nguyen, München (DE)

(21) Appl. No.: 18/566,899

(22) PCT Filed: Jun. 10, 2022
(86) PCT No.: PCT/EP2022/065889

§ 371 Date: Dec. 4, 2023

(30) Foreign Application Priority Data
Jun. 11, 2021 (EP) 21179080.3

(51) Int. Cl. G01N 33/543; G01N 33/532; G01N 33/58

(52) U.S. Cl. CPC G01N 33/54388; G01N 33/532; G01N 33/581; G01N 33/582

(57) ABSTRACT

The invention relates to a labeling DNA nanostructure for providing a signal amplifying detectable label in immunoassays, a test device, in particular a lateral flow test device, which comprises the labeling DNA nanostructure, a method for producing the labeling DNA nanostructure or the test device.

The present invention relates to a labeling nanostructure for providing a signal amplifying detectable label in immunoassays, in particular in bead-based immunoassays and in immunoassays comprising a porous membrane. The invention further relates to a test device, in particular to a lateral flow test device, which comprises the labeling nanostructure, and a method for producing the labeling nanostructure or the test device.

Commercial immunoassays, in particular lateral flow immunoassays (LFAs), as launched 1984 by Unipath, make a simple, yet very sophisticated system to detect and quantify an analyte. They are used for clinical analysis, the detection of toxins and pathogens as well as pesticides and drugs with a visual readout.

The advent of microfluidics has started the quest for novel immunoassay platforms and formats, which enable rapid immunoassay using the minimal volume of reagents. An example is the Optimiser ELISA by Siloam Biosciences, USA, which involves the conversion of the conventional 96-well MTP-based ELISA into microfluidic ELISA. The analysis time is only a few minutes as this hybrid system employs significantly reduced number of steps and a microfluidic immunoassay protocol. Similarly, a large number of prospective lab-on-a-chip technologies and formats have been developed toward the development of various microfluidic immunoassay, which are widely bead-based assay formats. In those assays, beads are coated with analyte specific antibodies. Biotinylated detection antibodies specific to the analyte of interest are added and form an antibody-antigen sandwich. A fluorescent label, e.g. phycoerythrin is conjugated to the detection antibodies via streptavidin and the fluorescent signal of the label is detected as it is in proportion to the amount of analyte bound to the bead. However, the detection signal of a single fluorophore is weak and the amount of analyte in a sample which can be detected thus limited. Therefore, there is a need for an amplification mechanism to reliably allow detecting of also lower analyte concentrations in a sample.

The most widely used microfluidic immunoassay format is the LFA. The LFA platform typically consists of a simple cellulose membrane, i.e., a porous membrane, where the analyte can migrate through and interact with molecules present in the membrane. The presence or absence of the analyte is visualized using a label, for example gold nanoparticles, dyed polystyrene, or latex beads. Labeled analytes accumulate upon migration by specific reactions to molecules, herein referred to as target or target reagents and/or control or control reagents already immobilized at a certain area on the membrane, named the capture zone or the binding area.

Very little sample volume and no sample preparation are required while the test result is provided after a few minutes. Low material cost and energy consumption are ecological factors driving their popularity as well. These advantages make LFAs virtually out of competition in the nanomolar concentration regime.

A major drawback and the reason why the polymerase chain reaction (PCR) test remains today's standard is that LFAs are not applicable for low analyte concentrations, i.e., in the picomolar regime. The reliability level of LFAs decreases with decreasing analyte concentration. A use of LFAs in the picomolar regime would give a high rate of false-negative results. The reliability at high analyte concentrations however has little benefit, i.e., value when at that point the disease has progressed too far. Yet, our healthcare system would greatly benefit from the diagnosis of infections and diseases at an early stage via LFA. Especially the work of global health programs is currently impeded because such tests are still unavailable. Furthermore, the PCR is a technology not accessible in the developing world due to a poor infrastructure and limited resources. Such technologies usually require maintenance, calibration of instruments, refrigeration of chemicals and reagents, and trained personnel. Another trivial seeming but determinant factor is the time-consuming transportation of sample from the medical office to the laboratory. Results are not available at the “point of care”. Having the test result within minutes relieves our health system as well as our individual life standard. Paper-based test strips, are stable, portable, require little user input and no laboratory, while giving immediate results.

Therefore, tuning the sensitivity of immunoassays, in particular of LFAs, to an appropriate level which ensures reliability, i.e., to a high analytical performance, addresses a real-world problem.

The sensitivity of LFAs is simply limited by the visibility of their labels. Conventionally one analyte molecule can bind to one label. Labels that reach a threshold number become visible for the human eye. Hence Increasing the number of the labels per analyte is therefore a desired technical goal for achieving an increased signal amplification upon detection. The document U.S. Pat. No. 9,651,549 B2 describes a method of employing a DNA dendrimer for signal amplification in immunoassays comprising from about 10 to about 1500 detectable labels. The synthesis of DNA dendrimers inherently leads to nanostructures of non-precisely known number of binding sites of the labels as the dendrimer structure is assembled from double stranded monomers added to a dendrimer structure layer by layer. Therefore, the sensitivity of a resulting immunoassay can only roughly be predetermined, and a quantitative analysis of a detection signal is thus prima facie excluded.

It is therefore an object of the present invention to provide a labeling nanostructure usable for immunoassays for coupling to an analyte, which allows for an improved control on the sensitivity of the immunoassay.

It is a further object of the invention to provide a test device for running an immunoassay, a method of producing the labeling nanostructure, a method of producing the test device, which utilizes the labeling nanostructure and a method for providing signal amplification to an immunoassay method, which uses the labeling nanostructure.

This object is attained by a labeling nanostructure according to the combination of features of independent claim 1, by the test device of claim 8 or the method of claim 12 of producing the labeling nanostructure or the method of claim 13 for producing the test device or the method of claim 14 for providing signal amplification to an immunoassay method or the use of claim 15 of using the labeling nanostructure.

Various preferred embodiments presented are particularly provided by the teachings of the dependent claims.

According to a first aspect, there is provided a labeling nanostructure as claimed in claim 1. The labeling nanostructure comprises a polynucleotide-based nanostructure, the polynucleotide-based nanostructure having a number of first and/or second binding sites for binding, in particular selectively, the polynucleotide-based nanostructure to an analyte. The labeling nanostructure, in particular the polynucleotide-based nanostructure, further comprises a predefined number N>1 of second binding sites, wherein each second binding site is configured to, in particular selectively, bind a detectable label, e.g., a molecule and/or a nanoparticle to the nanostructure. The number N of second binding sites is defined by a predefined sequence of nucleotides.

The invention generally relates to the use in immunoassay methods to amplify a detection signal of the respective immunoassay method by means of the labeling nanostructure, which acts as a label. An immunoassay is a biochemical test that measures the presence or concentration of a macromolecule or a small molecule in a solution through, in particular the use of an antibody or an antigen. In the immunoassay methods, the molecule is detected by detecting the label, typically by means of optically detecting the label. The molecule detected by the immunoassay is referred to as an “analyte” and is in many cases a protein, although it may be other kinds of molecules, of different sizes and types, as long as the proper antibodies that have the required properties for the assay are developed. Analytes in biological liquids such as serum or urine are frequently measured using immunoassays for medical and research purposes. Immunoassays come in many different formats and variations. Immunoassays may be run in multiple steps with reagents being added and washed away or separated at different points in the assay. Multi-step assays are often called separation immunoassays or heterogeneous immunoassays. Some immunoassays can be carried out simply by mixing the reagents and sample and making a physical measurement. Such assays are called homogeneous immunoassays, or less frequently non-separation immunoassays.

The labeling nanostructure according to the invention and respective methods relating to the labeling nanostructure, can be used as such a label in many different kinds of immunoassays, e.g., enzyme-linked immunosorbent assay, e.g. performed in a laboratory, in a well plate format (ELISA), lateral flow-based assays (LFA), e.g. performed outside a laboratory, on a porous membrane. Therefore, to serve as a label in a respective immunoassay method, the labeling nanostructure is configured to be bond to a conjugate, in particular to an immunoconjugate, e.g., an antibody, via the one or more first binding sites of the polynucleotide-based nanostructure, whereas the conjugate is capable to further bind to an analyte to be detected by a respective immunoassay method. The analyte then typically further binds to an antibody which immobilizes the analyte to a surface. The surface can be that of a porous membrane, thereby immobilizing the labeling nanostructure at a binding area of the porous membrane, in case of an LFA. However, the surface can be that of a particle, e.g., the surface of a bead, thereby immobilizing the labeling nanostructure at a binding area of the bead, in case of a bead-based immunoassay method. The bead can be, e.g. a magnetic or a fluorescent bead. A bead hereby refers to a microsphere, typically comprising diameters ranging from the nanometer to the micrometer range, e.g. from 100 nm-100 μm, 500 nm-50 μm, 800 nm-20 μm, 500 nm-10 μm, 500 nm-5 μm, 800 nm-2 μm.

In the following general and specific description, the application of the labeling nanostructure according to the invention is exemplarily described to those in the field of LFA. However, the skilled person understands that the application of the claimed labeling nanostructure can also be transferred to other immunoassays, in particular to those mentioned above.

The term “polynucleotide-based nanostructure” means, in particular, that the nanostructure comprises at least a portion being a polynucleotide or that the nanostructure is, substantially, formed by a polynucleotide.

The term “predefined number N>1 of second binding sites” is to be understood in such a way that the number of second binding sites of the labeling nanostructure is predetermined by selection of a nucleotide sequence of the polynucleotide-based nanostructure, in particular before the polynucleotide-based nanostructure is obtained, e.g., from synthesis.

Thereby, the selection of each respective nucleotide sequence, which relates to a binding site, is made such that binding criteria for each respective second binding site are fulfilled in order to bind a detectable label to the then obtained polynucleotide-based nanostructure, in particular one detectable label at each second binding site. These binding criteria may be application-specific criteria. For example, the individual bonds should be chemically predefined. For example, to chemically suit a specific binding site reagent attached to a detectable label, e.g., attached to the surface of a nanoparticle, to functionalize the nanoparticle for use as a detectable label. Further criteria are, for example, related to geometrical space requirements, such as positioning of the second binding sites at the nanostructure in such a way that binding of a detectable label to a second binding site adjacent to detectable label already bound to a second binding site can take place spatially. Meaning, that an unoccupied binding site at the nanostructure is not covered by the spatial dimension of a functionalized nanoparticle bound to a further binding site, in order to avoid that the unoccupied second binding site becomes inaccessible for further binding. In this respect, the predefined number N>1 of second binding sites relate to predetermined positions of second binding sites on the nanostructure while considering the geometries, e.g., the spatial extent, of the detectable labels attached to the polynucleotide-based nanostructure, such as their diameter, thereby avoiding inaccessibility of second binding sites, which in particular are adjacent to each other. For example, in such a way that the detectable labels do not interfere with each other, e.g., by the spatial extent of a tethered detectable label obscuring unoccupied binding sites, or tethered detectable labels pushing each other away. Further application specific criteria relate to, e.g., spatially distributing the binding sites to be present on certain areas of the nanostructure, in order to fulfill application-specific requirements. The skilled person can check the binding of the detectable label to the second binding sites of the nanostructure, for example, by means of conventional electron microscopy, for example after determining the polynucleotide sequences relevant for the binding site and synthesizing the nanostructure accordingly. He can also use the sensitivity of the detection signal to check whether it corresponds to the sensitivity derived from the number of binding sites by measuring known analyte concentrations.

Because the binding sites, in particular, the number N of second binding sites are predefined, the binding sites can be arranged on the surface of the nanostructure such that it is basically ensured that each detectable label can bind to a second binding site. Therefore, the number of binding sites essentially corresponds to the number of detectable labels. As each detectable label bound to the nanostructure contributes to an overall signal of the labeling nanostructure, when used in an immunoassay, the more detectable labels are bound to each site of the labeling nanostructure, the stronger the signal per labeling nanostructure. The labeling nanostructure according to the invention has the effect to linearly amplify a detection signal. The linear gain results from the fact that the number of second binding sites per polynucleotide-based nanostructure is the same for all polynucleotide-based nanostructures or for a group of polynucleotide-based nanostructures. In other words, the number of second binding sites per polynucleotide-based nanostructure is reproducible for each of the polynucleotide-based nanostructures of a group. In a respective immunoassay one group or different groups of polynucleotide-based nanostructures may be used, wherein within a respective group the number of second binding sites remains constant. The number of second binding sites determines the gain and can be chosen according to the requirements of the application by predefining the number N>1 of second binding sites.

Typically, each detection method has a lower detection limit below which a signal can no longer be detected by the immunoassay method. Thus, depending on the detection method of the immunoassay, a certain signal strength above the respective detection limit is required. An immunoassay should be able to detect the lowest possible concentration of an analyte. Since a single analyte binds to a single labeling nanostructure, the labeling nanostructure ideally provides a signal, that is above the detection limit of the immunoassay. However, since the number of binding sites per labeling nanostructure can be reproducibly controlled for all nanostructures, each labeling nanostructure contributes equally to the detection signal of the immunoassay and thus, depending on the amplification per nanostructure, even a few of the nanostructures might be sufficient to be above a detection limit of a particular immunoassay. The detection signal therefore scales linearly with the number of analyte molecules. The amplification per labeling nanostructure scales with the number of detectable labels, i.e. the number of binding sites at the nanostructure. The sensitivity, which is the slope of the detection signal as a function of the analyte concentration, can thus be advantageously adjusted, i.e., predetermined by the number of binding sites of the labeling nanostructure. Further, because the sensitivity is known, i.e., predetermined through the number of binding sites, also a quantitative analysis of the detection signal is possible and therefore, an absolute analyte concentration can be easily calculated without the need of a calibration curve.

A nanostructure hereby relates to a polynucleotide-based structural unit, e.g., an architecture of—two—dimensional or three—dimensional shapes from polynucleotides comprised of 2 or more, in particular up to 350, 1000 or even 10000 nucleotides and/or oligonucleotides that, in particular through base pair recognition, assemble into an object of defined shape and size, e.g., a DNA Origami as a particular instance of an object having a defined shape and size, whereas the desired shape and size is obtained from the folding route of a given scaffold and a respective set of staple sequences that can fulfill the folding. The shape is thus predetermined by the sequences of the nucleotides and/or oligonucleotides that, as a whole or in part, recognize each other and bind to each other to form the nanostructure. The size is limited by the length of the scaffold used for folding, i.e., the number of nucleotides and/or polynucleotides used. In some instances, the shape may be described means an aspect ratio, e.g., for a rod-shaped nanostructure, the length:width:height ratio. The polynucleotide-based nanostructure preferably has a molecular mass, which preferably is chosen from the following preferred ranges: 100 kilodalton (kDa) to 100 megadalton (MDa), 250 kDa to 50 MDa, 500 kDa to 20 MDa, 1 to 10 MDa; another preferred range is between 4 to 6 MDa.

Polynucleotide-based structural units include but are not limited to scaffolded deoxyribonucleic acid (DNA) origami, scaffolded ribonucleic (RNA) origami, scaffolded hybrid DNA: RNA origami, scaffold free DNA single—stranded tile (DNA brick) systems, scaffold—free multi—stranded DNA tile systems or RNA tile systems, intramolecularly-folded single—stranded RNA or single—stranded DNA origami, or any DNA/RNA analogues, e.g., LNA, PNA or XNA. Nucleic acid analogues are compounds which are analogous, i.e., structurally similar, to naturally occurring RNA and DNA, used in medicine and in molecular biology research. Typically, the analogue nucleobases confer, among other things, different base pairing and base stacking properties. Examples include universal bases, which can pair with all four canonical bases, and phosphate-sugar backbone analogues such as PNA, which affect the properties of the chain, e.g., PNA can even form a triple helix. Nucleic acid analogues are also called Xeno Nucleic Acid. Artificial nucleic acids include peptide nucleic acid (PNA), Morpholino and locked nucleic acid (LNA), as well as glycol nucleic acid (GNA), threose nucleic acid (TNA) and hexitol nucleic acids (HNA). Each of these is distinguished from naturally occurring DNA or RNA by changes to the backbone of the molecule.

The term “detectable” is to be understood as meaning such that the presence of a label and/or a plurality of labels attached to the nanostructure can be measured, e.g., by visual and/or optical detection, in particular by fluorescence detection. In this respect a label is detectable.

A detectable label hereby further relates to a molecule and/or a nanoparticle, as a nanometer sized structure. In case of a nanoparticle as the detectable label, the nanoparticle is functionalized. A functionalized nanoparticle hereby refers to a nanoparticle, which has a functional substituent attached to it. The function of the functional substituent is to bind the nanoparticle to the second binding site but may also be related to the effect of preventing aggregation of the functionalized nanoparticle in solution. The substituent is linked to the nanoparticle's surface. Linkage of the substituent to the nanoparticle's surface is achieved at least through a binding group. The binding group at least forms a bond with the substituent and with the surface of the nanoparticle. In a particular embodiment the binding group forms a bond with a silver surface of the nanoparticle. The binding group used for attaching the substituent to the nanoparticle can be a thiol moiety of the form R-SH. In a preferred embodiment of a detectable label, the substituent is bond to the silver surface of the nanoparticle via sulfur.

The detectable labels can be chosen from the group of gold nanoparticles, silver nanoparticles, metal nanoparticles, dyed polystyrene or latex beads, enzyme or fluorophores, i.e., fluorescent labels, e.g., fluorescent particles, a Q-Dot, a carbon nanotube, a silver coated particle, a cellulose particle.

The substituent can be any molecule. The substituent preferably further comprises a functional group, e.g., a carboxyl group, an amino acid, a protein, an antibody, a virus or a hormone.

The first and/or second binding sites comprise within the polynucleotide-based nanostructure respective different nucleotide or oligonucleotide sequences. Thereby, each respective binding site can individually be “programmed”, i.e., predefined by an unambiguously assignable nucleotide and/or oligonucleotide sequence. During preparation of the nanostructure, these unambiguously assignable nucleotide and/or oligonucleotide sequences are included into a polynucleotide of the nanostructure. A part of these unambiguously assignable sequences peeks out of the prepared nanostructure. Other nucleotides and/or oligonucleotides can be attached to this part or this part is chemically modified with a desired binding reagent further comprised by the binding site to link the nanostructure to a detectable label or to an analyte or to a target reagent.

As used herein, a “binding reagent” refers to any substance that binds to a target, a detectable nanoparticle or an analyte with desired affinity and/or specificity. In this sense, a binding reagent is used in analogy to a substituent within the present description of the invention.

The binding reagents comprise molecules directly selected from the group of DNA-sequences, or RNA or PNA sequences or molecules for modification of the part of the sequences which peeks out of the nanostructure, selected from the group of fluorescein isothiocyanate (FITC), digoxigenin (Dig), biotin, or selected from the group of thiol- or amino groups, or selected from the group of proteins, e.g., streptavidin, antibodies.

Non-limiting further examples of the binding reagent include cells, cellular organelles, viruses, particles, microparticles, molecules, or an aggregate or complex thereof, or an aggregate or complex of molecules. Exemplary binding reagents can be an amino acid, a peptide, a protein, e.g., an antibody or receptor, a nucleoside, a nucleotide, an oligonucleotide, a nucleic acid, e.g., DNA or RNA, a vitamin, a monosaccharide, an oligosaccharide, a carbohydrate, a lipid, an aptamer and a complex thereof.

As used herein, the term “antibody” is an immunoglobulin molecule capable of specific binding to an analyte, such as a carbohydrate, polynucleotide, lipid, polypeptide, etc., through at least one antigen recognition site, located in the variable region of the immunoglobulin molecule, and can be an immunoglobulin of any class, e.g., IgG, IgM, IgA, Ig) and IgE. IgY, which is the major antibody type in avian species such as chicken, is also included within the definition. As used herein, the term encompasses not only intact polyclonal or monoclonal antibodies, but also fragments thereof (such as Fab, Fab′, or F(ab′)2, Fv), single chain (Sclv), mutants thereof, naturally occurring variants, fusion proteins comprising an antibody portion with an antigen recognition site of the required specificity, humanized antibodies, chimeric antibodies, and any other modified configuration of the immunoglobulin molecule that comprises an antigen recognition site of the required specificity.

As used herein, the term “antigen” refers to a molecule that is specifically bound by an antibody through its antigen recognition site. The antigen may be monovalent or polyvalent, i.e., it may have one or more epitopes recognized by one or more antibodies. Examples of kinds of antigens that can be recognized by antibodies include polypeptides, oligosaccharides, glycoproteins, polynucleotides, lipids, etc.

As used herein, the term “specifically binds” refers to the specificity of a binding reagent, e.g., an antibody, such that it preferentially binds to a defined analyte or target. Recognition by a binding reagent or an antibody of a particular analyte or target in the presence of other potential interfering substance(s) is one characteristic of such binding. In some embodiments, a binding reagent that specifically binds to an analyte avoids binding to other interfering moiety or moieties in the sample to be tested.

The labeling nanostructure further comprising third binding sites, wherein the third binding sites comprise a third binding reagent to specifically bind the labeling nanostructure to a control reagent. The third binding agent is selected from the group of DNA-sequences, or RNA or PNA sequences or molecules for modification of the part of the sequences, which peeks out of the nanostructure, selected from the group of fluorescein isothiocyanate (FITC), digoxigenin (Dig), biotin, or selected from the group of thiol- or amino groups, or selected from the group of proteins, e.g., streptavidin, antibodies. The binding reagent of the third binding site is selected in a way that it specifically binds to a substance, e.g., to an immobilized molecule, such that binding occurs exclusively with this substance or molecule at the third binding site.

As used herein, a “control reagent” refers to any substance that binds to the third binding site directly and/or to a binding reagent of the third binding site, e.g., a molecule thereof, with desired affinity and/or specificity, to differentiate between labeling nanostructures with an analyte being bound to it and those without an analyte being bound to it, e.g., excess nanostructures. The control reagents thus serve to ensure proper functioning of a test device using lateral flow, e.g., to indicate proper transport through a porous membrane.

As used herein, the term “porous” refers to porosity or void fraction as a measure of the void spaces in a material and is a fraction of the volume of voids over the total volume, between 0 and 1, or as a percentage between 0% and 100%. It also refers to the “accessible void”, the total amount of void space accessible from the surface of a substrate. E.g., when a droplet containing the nanostructures is applied onto the porous surface of the substrate, the porosity allows the nanostructures to migrate from the porous surface into the pores of the substrate and through the pores by capillary forces via the accessible voids, i.e., accessible to the labeling nanostructure. Thereby, the pores allow transport of the labeling nanostructure through the substrate. A substrate may comprise a porous membrane. In this sense, a porous membrane is a selective barrier. It allows some things to pass through but stops others. Such things may be molecules, ions, or other small particles, e.g., nanoparticles, or nano-sized objects, or the labeling nanostructure.

Further advantageous embodiments of the labeling nanostructure according to the invention have been specified in the dependent claims 2 to 7.

Embodiments are described or can be gathered from the description, which can be arbitrarily combined with each other or with other aspects of the present invention, unless such combination is explicitly excluded or technically impossible.

According to a first embodiment of the labeling nanostructure, the polynucleotide-based nanostructure has a predefined size and/or a predefined shape.

The terms “size” and/or “shape” of the nanostructure hereby relate to the—two-dimensional or three—dimensional overall shape, i.e., the spatial extension of the labeling nanostructure in analogy to a protein conformation, wherein the spatial arrangement of the polynucleotides and/or oligonucleotides, is obtained in particular through base pair recognition, which assembles the polynucleotides into this object of defined shape and size, thereby determining the overall shape of the object.

E.g., a DNA Origami as a particular instance of an object having a defined shape and size, whereas the desired shape and size is obtained from the folding route of a given scaffold and a respective staple sequence that can fulfill the folding.

In this respect, the shape is predetermined by the sequences of the nucleotides and/or oligonucleotides that, as a whole or in part, recognize each other and bind to each other to form the nanostructure.

In this respect, the size is limited by the length of the scaffold used for folding, i.e., the number of nucleotides and/or polynucleotides used. In some instances, the shape may be described means an aspect ratio, e.g., for a rod-shaped nanostructure, the length:width:height ratio.

Sine the shape and/or size is predefined, the nanostructure can be configured to a specific porous membrane, to ensure that the porous membrane does not become clogged by the multiple nanostructures during transport. This is of importance when, a high concentration of nanostructures is to be transported through a membrane of a certain pore size, and the detectable labels are formed from nanoparticles with a large diameter.

According to a second embodiment of the labeling nanostructure, wherein
a) the labeling nanostructure consists of the polynucleotide-based nanostructure, or
b) the labeling nanostructure comprises at least one further subcomponent, the at least one further subcomponent having, in particular, a predefined size and/or a predefined shape or not having a predefined size and/or a predefined shape.

A subcomponent refers to a nanosized object, e.g. the nanostructure or a combination of a plurality of nanostructures linked to each other to form a larger sized labeling object. This beneficially allows improving the signal amplification to even higher gain factors.

According to a third embodiment of the labeling nanostructure, wherein the polynucleotide-based nanostructure comprises one or more scaffold strands.

A scaffold strand hereby relates to a long polynucleotide strand, e.g., a long single-stranded DNA strand, typically comprising 7000 nucleotides. In case of a DNA Origami, the scaffold strand comprises hundreds of designed short single-stranded DNAs as staples. Each staple has multiple binding domains that bind and bring together otherwise distant regions of the scaffold via crossover base pairing, thereby folding the scaffold in a predefined manner. As the staple sequences can be predefined, the geometry, e.g., the size and shape of the resulting nanostructures is advantageously predeterminable and can thus be adapted to an application specific requirement, e.g., an application specific parameter, as the mesh size of a porous membrane of a lateral flow test device.

According to a fourth embodiment of the labeling nanostructure, wherein the predefined number N of second binding sites are configured to bind a label, i.e. a detectable label, suitable for being detected in an immunoassay, the label being selected from the group of preferred labels comprising an enzyme or a fluorescent label, or a particle label, in particular a visible or a non-visible particle label, e.g. a fluorescent particle, a liposome, a gold particle, a latex particle, a Q-Dot, a carbon nanotube, a silver particle and a silver coated particle, a cellulose particle.

According to a fifth embodiment of the labeling nanostructure, wherein the predefined number N preferably is 1<N<1000, preferably 10<=N<1000, preferably 10<=N<750, preferably 10<=N<500, preferably 10<=N<400, preferably 10<=N<350, preferably 10<=N<300. Preferably, N>2; preferably, N>3, preferably, N>4, preferably, N>5, preferably, N>6, preferably, N>7, preferably, N>8, preferably, N>9, preferably, N>10. A number of already N=2 achieves a useful amplification in comparison with N=1. A number of N>=5, in particular N>=10 achieves a remarkable amplification most useful for replacing a single label in an established immunoassay by the labeling nanostructure according to the invention.

According to a sixth embodiment of the labeling nanostructure, wherein each second binding site is adapted for binding a detectable label, whose detection produces a first signal having a predefined first value V1, and wherein the predefined number N of second binding sites provide a sum of N detectable labels, whose detection produces a second signal having a predefined second value V2, which is a known amplification Amp of the first value, i.e., V2=Amp*V1.

Because the second signal is a known amplification of the first signal V1, a linear relationship allows simple calibration, e.g., of a lateral flow test device and sensitivity of this device remains constant over the entire measurement range.

In some case, each detectable label produces a first signal S1 upon detection, and wherein the sum of detectable labels provided by the number N of second binding sites produces a second signal S2 upon detection, which relates to the first signal as S2=N*F*S1, wherein F describes a binding occupancy at a binding site. The binding occupancy F varies between 0.8 to 1.2 or between 0.9 to 1.2 or between 0.9 to 1.1.

According to a specific further embodiment, wherein the labeling nanostructure comprises a number N of detectable labels, in each case one detectable label is bound to each of the predefined number N of second binding sites. In this embodiment, the binding occupancy F is one.

According to a seventh embodiment, the detectable label comprises a functionalized nanoparticle, having a metal core, a silver coating, and a sulfide bond substituent.

The silver coating of each of the functionalized nanoparticles forms a shell around the metal core and the metal core is at least partially covered by the silver shell. This embodiment has the advantage, that under resonant excitation, metal nanoparticles have the unique ability to concentrate the free-space optical field within subwavelength regions, wherein this ability is based on surface plasmon excitation. The overall plasmonic behavior of gold (Au) and silver (Ag) nanoparticles is similar, however, silver is known to give higher field effects due to a lower plasmon damping leading to more intriguing optical properties and thus to enhanced optical performances.

The detectable labels can be further chosen for each embodiment from the group of gold nanoparticles, Ag nanoparticles, metal nanoparticles, dyed polystyrene, or latex beads. In a further embodiment it is also possible to combine different types of detectable labels at one polynucleotide-based nanostructure

According to a second aspect, a test device for running an immunoassay, in particular an LFA, is provided. The test device comprises a test substrate, in particular a porous substrate, comprising a plurality of labeling nanostructures as previously described.

The LFA typically consists of a simple cellulose membrane, i.e., a porous membrane, where the analyte can migrate through and interact with molecules present in the membrane. The presence or absence of the analyte is visualized using the detectable labels, for example gold nanoparticles, Ag nanoparticles, metal nanoparticles, dyed polystyrene, or latex beads, which are attached to the polynucleotide-based nanostructure at the specified second binding sites to form the labeling nanostructure.

The analytes specifically bind to the first binding sites of the labeling nanostructure.

In one embodiment of the test device, the detectable labels are bond to the second binding sites of the labeling nanostructure when the labeling nanostructure is applied to the test substrate of the test device.

In a further embodiment, the detectable labels are not bond to the second binding sites of the labeling nanostructure when the labeling nanostructure is applied to the test substrate of the test device. In this embodiment, the detectable labels are separately provided.

Therefore, in a first step, the labeling nanostructures are applied to the test substrate of the test device. In a second step the detectable labels are applied to the test substrate of the test device.

In the first step a solution containing the analyte can be further added to the substrate after applying the labeling nanostructures to the test substrate. Alternatively, a solution comprising the analyte and the labeling nanostructure is applied.

In this case, the labeling nanostructures migrate through the porous membrane of the substrate with the analyte being attached to the respective first binding sites of the labeling nanostructures. In the second step, the detectable labels applied to the test substrate then migrate through the substrate until they bind, in particular, specifically to the second binding sites of the respective labeling nanostructures.

The invention also relates to a kit, whereas the kit comprises the test device with the substrate, wherein the substrate comprises the labeling nanostructures, and wherein the kit further comprises a solution containing the detectable labels.

The analyte specifically binds to molecules immobilized in the binding zone of the test substrate. Molecules hereby relate to those selected from the group of DNA-sequences, or RNA or PNA sequences or molecules for modification of the part of the sequences, which peeks out of the nanostructure, selected from the group of fluorescein isothiocyanate (FITC), digoxigenin (Dig), biotin, or selected from the group of thiol- or amino groups, or selected from the group of proteins, e.g., streptavidin, antibodies. The molecules at the binding zone for immobilizing the analyte are selected to selectively bind to the analyte.

The labeled analytes accumulate upon migration through the substrate, e.g., a membrane, by specific reactions to the molecules, herein referred to as target or target reagents and/or control or control reagents. The substrate preferably comprises a target zone and a control zone. In the target zone, the labeled analytes specifically bind to the target reagents. In the control zone, those nanostructures without an analyte, i.e., excess nanostructures, bind to the control reagent. The control zone is used to ensure that the test device is functioning properly. For example, that the porous membrane is not clogged, preventing migration of the nanostructures, thereby incorrectly displaying the test result.

The labeling nanostructure according to the invention, preferably, can be a nanoscale object, having in particular a length scale of between 1 to 100 nanometers, and/or may in particular include the features of a nanoscale object as defined in the international patent application PCT/EP2020/082866, in particular in claim 13 thereof, wherein the functionalized nanoparticle defined by claim 1 of said patent application may form a detectable label for the labeling nanostructure. A functionalized nanoparticle is preferably prepared according to the following method, whereas the functionalized nanoparticle comprises or consists of a metal core, a silver coating and a sulfide bond substituent, in an aqueous solution, the method comprising a step of chemical functionalization of a metal nanoparticle in the aqueous solution, wherein the aqueous solution comprises or consists of water and ingredients, wherein the ingredients comprise or consist of the metal nanoparticle, a thiol of the form R-SH, where R represents a substituent, and a silver compound, the substituent being, preferably, organic, and having, preferably, a functional group.

The functional group, respectively preferably, comprises or consists of a carboxyl group (—COOH), an aldehyde group (—CHO), a hydroxyl group (—OH), an amino group (—NH2), an amide group (—CONH), and/or wherein the substituent comprises a carboxyl group, an amino acid, a protein, an antibody, a virus or a hormone, or two or more thereof.

The substituent can be any molecule, which is capable of having a binding group (R-SH). Within the present description of the invention, the terms “substituent” and “ligand” have the same meaning, if not defined to the contrary. In particular, the thiol, which includes the substituent, comprises or consists of mercaptopropionic acid (MPA), mercapto methoxy polyethylene glycol (mPEG-SH), PEG-SH, or most preferably DNA-SH. Preferably, the thiol, which includes the substituent, comprises at least one of 2,5,8, 11, 14, 17,20-Heptaoxadocosane-22-thiol, or CH3O(CH2CH2O)nCH2CH2SH. The substituent preferably further comprises a functional group, e.g. carboxyl group, an amino acid, a protein, an antibody, a virus or a hormone.

The binding agent is attached to the silver surface, which covers the metal core nanoparticle through a chemical binding of the thiol-group (—SH). The binding agent is attached to the silver surface, which covers the metal core nanoparticle through chemically binding of the thiol-group (—SH) during the reaction, in particular during the wet chemical reaction. That is, during the silver deposition on the surface of the metal core nanoparticle binding of the substituents occurs. Upon this reaction a color change of the solution visibly occurs.

Thereby most preferably a sulfide bond is formed. Chemical binding comprises a covalent bond, an ionic bond or a coordinate bond.

Chemical functionalization refers to binding of the substituent onto the surface of the nanoparticle having a metal core.

The water of the aqueous solution preferably is a purified water, preferably a distilled water, most preferably a double-distilled water (abbreviated “ddH2O”). Storage of the functionalized nanoparticle according to the invention preferably takes place in aqueous solution, preferably in ddH2O, preferably in the absence of a buffer.

According to the method provided by the invention an aqueous solution comprising water and the essential ingredients of at least a metal nanoparticle, a thiol of the form R-SH and a silver compound is used. A wet chemical reaction is started. In particular upon adding a pH increasing ingredient to the aqueous solution the wet chemical reaction is initialized. Upon initiation of the reaction, silver of the silver compound is deposited on the surface of the metal nanoparticles. Further, the thiolated substituents and or those substituents comprising an SH-group attach to the silver.

The spectral properties, in particular the color of the functionalized nanoparticles, in particular when deposited on the test substrate, depend on the size and/or the geometry of the functionalized nanoparticles. Therefore, adjusting the size and/or the geometry of the functionalized nanoparticles may be used to provide functionalized nanoparticles of different color. In particular when using a chemical functionalization (of the functionalized nanoparticles) depending on the size and/or the geometry (of the functionalized nanoparticles), a multiplexing test method may be provided. The test method may be a multiplexing test method being configured to utilize different groups of functionalized nanoparticles, each group having a different color. A test device for performing a multiplexing test method may contain a first group of functionalized nanoparticles having a first characteristic size and/or geometry and additionally a second group of functionalized nanoparticles having a second characteristic size and/or geometry, the second size and/or geometry being different from the first size and/or geometry, and if more than two different visual markers are to be provided, even more groups of different functionalized nanoparticles having, respectively, differing size and/or geometry.

The test substrate may be a test strip. The test substrate may be a pad. The test, method, preferably, is a lateral flow test. According to an embodiment, representing a lateral flow test, the test method operates by running a liquid sample along the surface of a pad with reactive molecules. A pad may contain an open-porous material, in particular a series of capillary beds, such as pieces of porous paper, microstructured polymer, or sintered polymer. For the purpose of applying a lateral flow test according to a preferred embodiment of the test method, the pad may act as a sponge and be able to hold an excess of sample fluid. A pad, preferably, has the capacity to transport a sample fluid, in particular a medical body fluid (e.g., urine, blood, saliva) spontaneously. A pad may contain a stack including a first conjugate pad layer and a second conjugate pad layer.

Further advantageous embodiments of the test device have been specified, in particular, in the dependent claims 8 to 11.

According to a first embodiment of the test device, the plurality of labeling nanostructures comprises groups of labeling nanostructures, wherein the groups distinguish by their label sizes, wherein within each group a uniform label size prevails.

Thereby, the test device is advantageously able to perform several tests to identify an analyte or a number of different analytes in parallel. This type of multiplexing of individual testing procedures saves time and reduces the amount of test devices necessary for testing a sample probe, e.g., a solution containing an analyte of a group of different analytes. Detecting multiple analytes in a single test rather than using many individual tests is advantageous in situations where only a small sample volume is available in order to maximize its use. Further, to assist diagnosis where the presence of several markers, i.e., labels, together is required. It offers a cost-saving benefit to end-users.

According to a second embodiment of the test device, the test device is adapted for running a lateral flow immunoassay, wherein the test substrate comprises a strip including a porous membrane. The porous membrane of the strip contains the plurality of labeling nanostructures, whereas the labeling nanostructures comprise the ploynucleotide-based nanostructures with their first binding site having a first binding site reagent, which is capable to bind to an analyte to be detected by the immunoassay.

The strip may contain several pads, e.g., a sample pad, a conjugate pad, an incubation and a detection pad. The pad may act as a sponge and is able to hold an excess of sample fluid. A pad, preferably, has the capacity to transport a sample fluid, in particular a medical body fluid (e.g., urine, blood, saliva) spontaneously. In this respect, a pad may comprise a porous membrane. A pad may contain a stack including a first conjugate pad layer and a second conjugate pad layer. The analyte is applied to the sample pad. The conjugate pad typically contains the plurality of labeling nanostructures with the first binding reagents bound to the first binding sites. The first binding reagents are configured to bind to the analyte further specifically.

According to a third embodiment of the test device, the test substrate comprises a binding area, where target reagents are bound to the test substrate and are thereby immobilized at the substrate and a solution containing the analyte being labeled by the labeling nanostructures is capable to flow along the test substrate to the binding area, which is configured to, in particular selectively, bind the analyte the analyte, which is labeled by the labeling nanostructures to the immobilized target reagents.

According to a third aspect, a method of producing the labeling nanostructures is provided.

The method of producing the labeling nanostructure comprises the step of:
synthesizing the polynucleotide-based nanostructure based on a predefined sequence of nucleotides, including one or more first binding sites for binding the polynucleotide-based nanostructure to an analyte, and including a predefined number N>1 of second binding sites being defined by the predefined sequence of nucleotides, each second binding site being configured for binding a detectable label;

According to a first embodiment of the method of producing the labeling nanostructures, the method comprises the step of binding a detectable label to each of the predefined number N>1 of second binding sites.

According to a second embodiment of the method of producing the labeling nanostructures, the method comprises the step of binding a conjugate, in particular an immunoconjugate, e.g., an antibody, to the one or more first binding sites, the conjugate being capable of binding to an analyte to be detected by an immunoassay.

A conjugate generally refers to a compound formed by the joining of two or more chemical compounds, e.g., a metal nanoparticle functionalized with multiple antibodies. Further examples for a conjugate are a metal particle coated by proteins, a metal particle coated by DNA, an antibody with a fluorescent label. A conjugate as used herein in particular refers to a compound formed by the joining of a binding agent and a detectable label.

According to a fourth aspect, a method of producing the test device is provided.

The method of producing the test device comprises the steps of
providing the test substrate, and
applying to the test substrate the plurality of the labeling nanostructures.

Preferably, the plurality of labeling nanostructures is applied to a conjugate pad of the test substrate. In a first case, the plurality of labeling nanostructures is applied to the test substrate and provided to a user with the first and/or second binding sites being adapted to bind to a specific detectable label and/or to a specific analyte by having a respective specific binding reagent attached to them, e.g., a user preselects a desired type of a first and/or of a second binding reagent according to his specific application. This selected binding reagent is then attached to the labeling nanostructure. For example, the first binding sites of the labeling nanostructure comprise a streptavidin molecule, such that the user can bind with his, e.g., biotinylated antibodies to the first binding site via a streptavidin—biotin binding complex.

Alternatively, in a second case, the plurality of labeling nanostructures is applied to a test substrate and is provided to the user with the first and/or second binding sites being not yet adapted to a, e.g., user specific detectable label and/or to a user specific analyte. In this case, there is no binding reagent attached to the first and/or second binding sites, respectively. Therefore, the respective binding sites must be provided with a respective binding reagent by the user, which advantageously allows the user to store and to individually adapt the labeling nanostructures in short time and according to his specific application individually. A user may therefore add a solution containing the respective specific binding reagents to the test substrate.

According to a fifth aspect, a method for providing signal amplification to an immunoassay method is provided.

The method for providing signal amplification to an immunoassay method, wherein the immunoassay method uses a single detectable label, and wherein the method for providing signal amplification provides the following steps:
providing the labeling nanostructure as claimed for acting as an amplifier,
replacing and/or adding, in the immunoassay method, the single detectable label by the labeling nanostructure as claimed, which comprises a number N>1 of detectable labels.

The single detectable labels can be replaced by the labeling nanostructure and or the labeling nanostructure is added to the existing immunoassay method using the single detectable labels. In this case, the detectable labels of the nanostructure preferably differ from the single detectable labels, e.g., the single detectable labels comprise fluorescent nanoparticles, wherein the detectable labels of the nanostructure comprise functionalized metal nanoparticles. Both types of detectable labels are thus detectable by different detection methods. Replacing is hereby understood as to substitute the single detectable label used in the immunoassay method, with the labeling nanostructure having a number of N>1 detectable label.

According to a further aspect, the labeling nanostructure is used for acting as a signal amplified label in an immunoassay, for example in a lateral flow immunoassay, or in a bead based immunoassay.

Further advantages, features and applications of the present invention are provided in the following detailed description of the exemplary embodiments and the appended figures. The same components of the exemplary embodiments are substantially characterized by the same reference signs, except if referred to otherwise or if other reference signs emerge from the context. In detail:

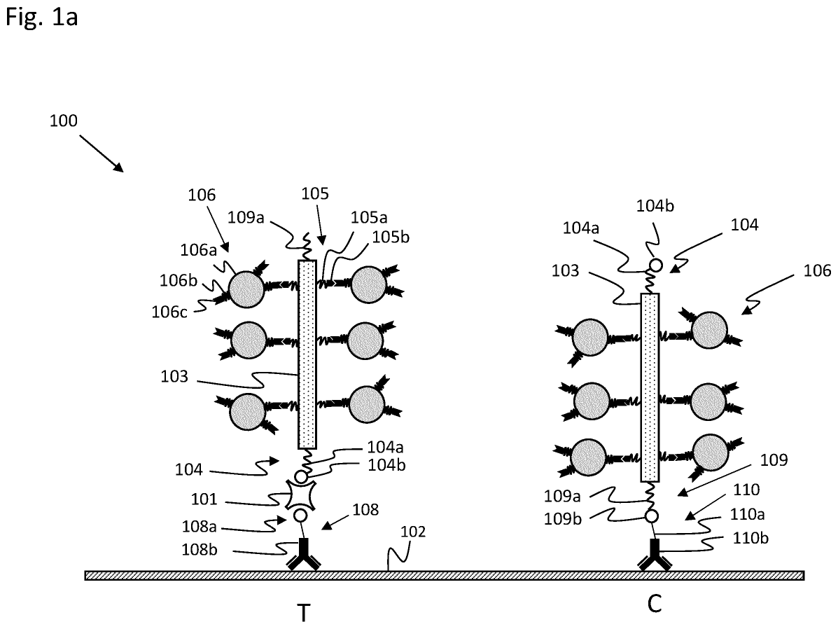

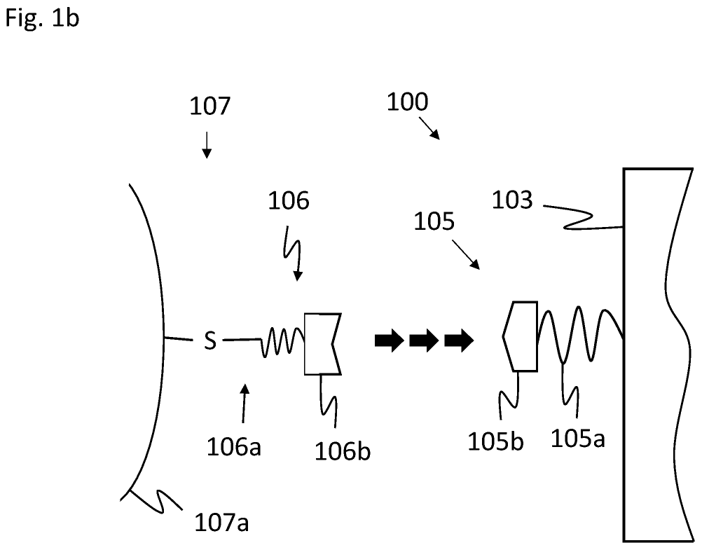

FIG. 1a shows an embodiment of two immobilized labeling nanostructures, wherein the one is labeled with functionalized nanoparticles and attached to a target molecule via an analyte (left) and wherein the other is attached to a control molecule via a third binding site of the nanostructure (right).

FIG. 1b shows an embodiment of a detectable label, configured to react with a second binding site of the labeling nanostructure.

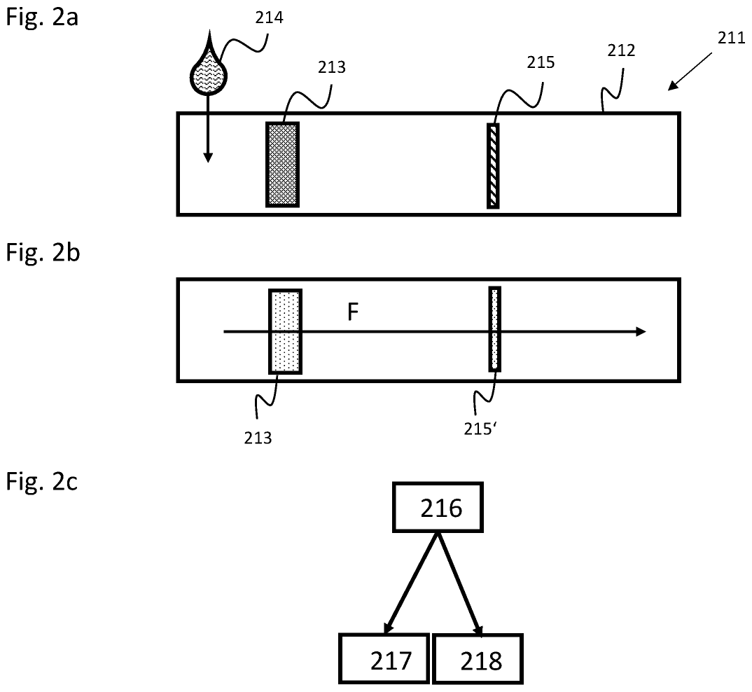

FIG. 2a shows an embodiment of a test device for performing a lateral flow test according to the invention, in a first status during application.

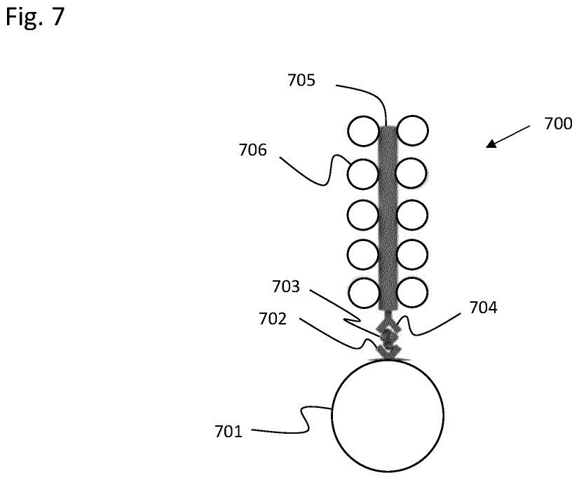

FIG. 2b shows the test device of FIG. 7a, in a second status during application.

FIG. 2c shows a diagram describing the method of producing a test device for performing a lateral flow test.

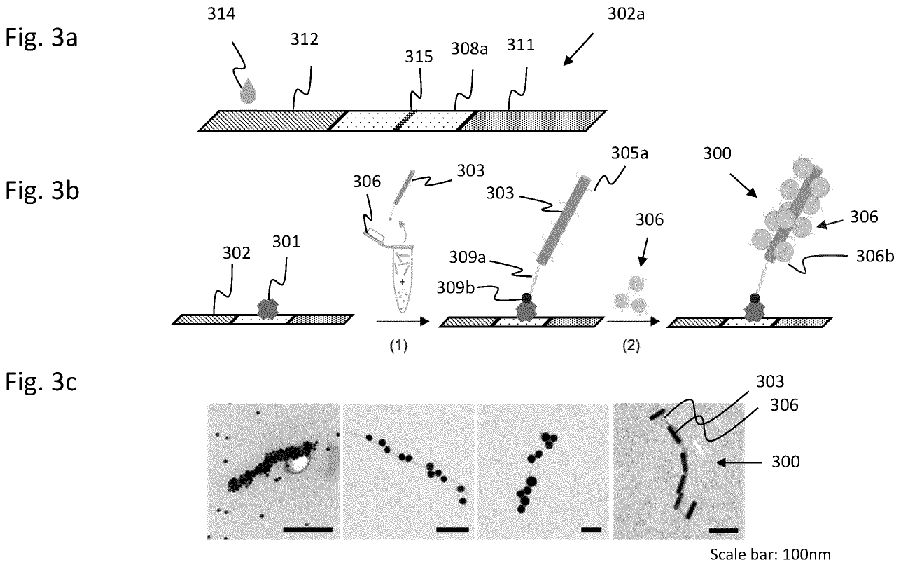

FIG. 3a shows an embodiment of a paper-based lateral flow assay schematically.

FIG. 3b shows the scheme of the lateral flow assay of FIG. 3a using a DNA origami, having a 6-helix-bundle.

FIG. 3c shows TEM micrographs of the 6-helix-bundle of FIG. 3b conjugated to (from left right) 10, 20, 40 nm gold nanoparticles and 65×15 nm gold nanoparticles (scale bars are 100 nm).

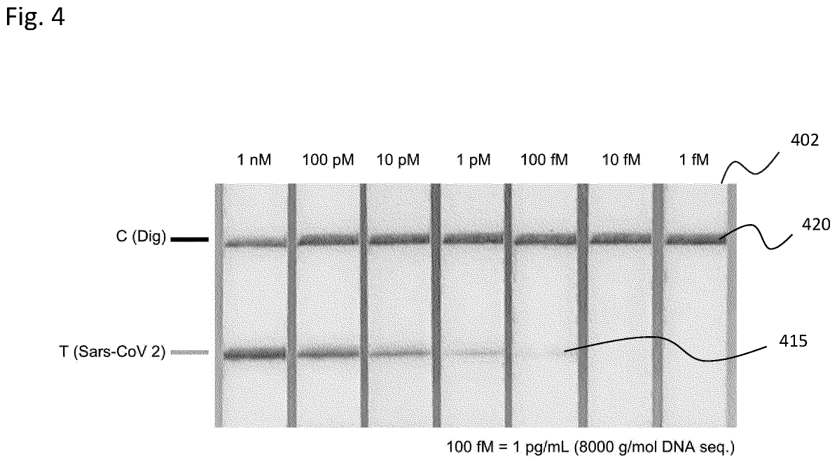

FIG. 4 presents test results of a lateral flow immunoassay with analyte concentrations ranging from 1 nM to 1 fM.

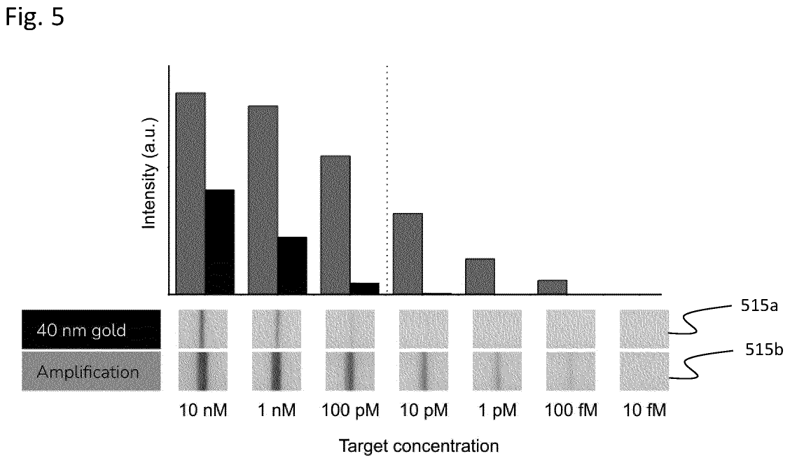

FIG. 5 shows test lines and corresponding intensity plots of a conventional lateral flow test using 40 nm gold nanoparticle compared to a lateral flow test using detectable nanostructures as claimed.

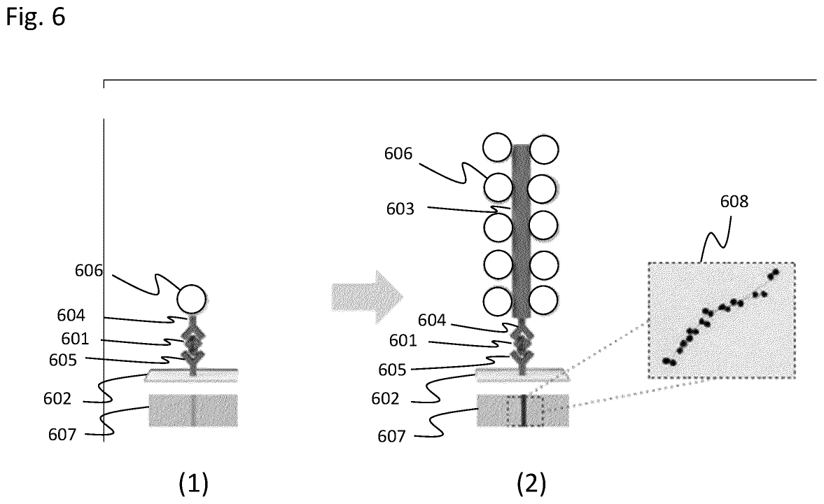

FIG. 6 shows an embodiment of the method for providing signal amplification to an immunoassay, which uses a single detectable label.

FIG. 7 shows an embodiment of the labeling nanostructure attached to a bead, for use in a bead-based immunoassay method.

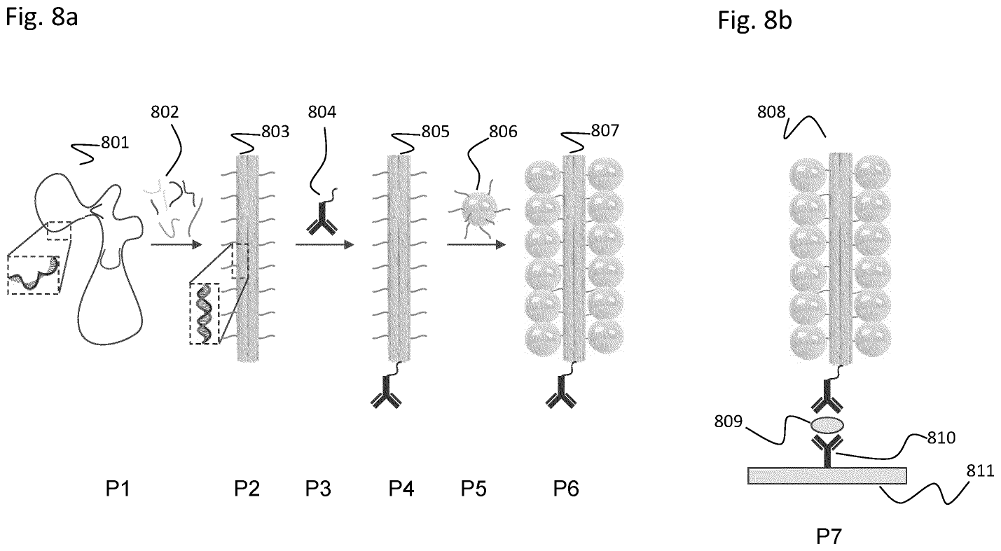

FIG. 8a shows a method of producing an inventive labeling nanostructure, according to an embodiment of the invention.

FIG. 8b shows an embodiment of applying the labeling nanostructure of FIG. 8a in an immunoassay.

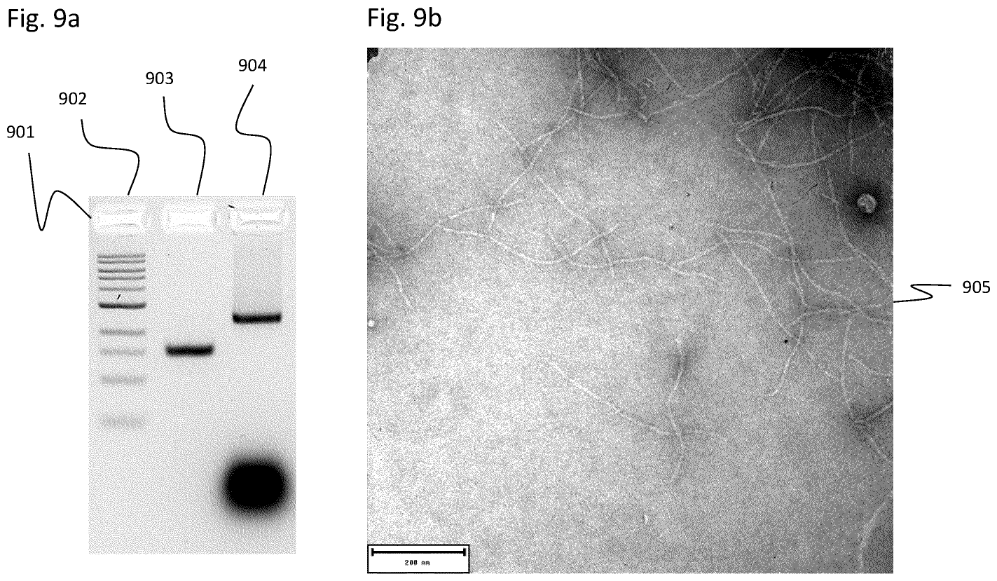

FIG. 9a shows experimental results from gel electrophoresis for demonstrating the synthesis of the polynucleotide nanostructure formed in FIG. 8a.

FIG. 9b shows experimental results from transmission electron micrography for demonstrating the synthesis of the polynucleotide nanostructure formed in FIG. 8a.

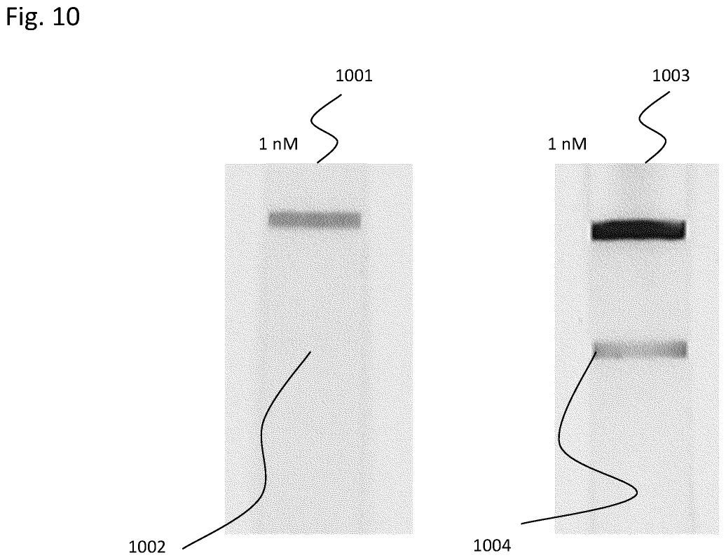

FIG. 10 shows the application of the polynucleotide nanostructure formed in FIG. 8a, in a lateral flow immunoassay in sandwich format.

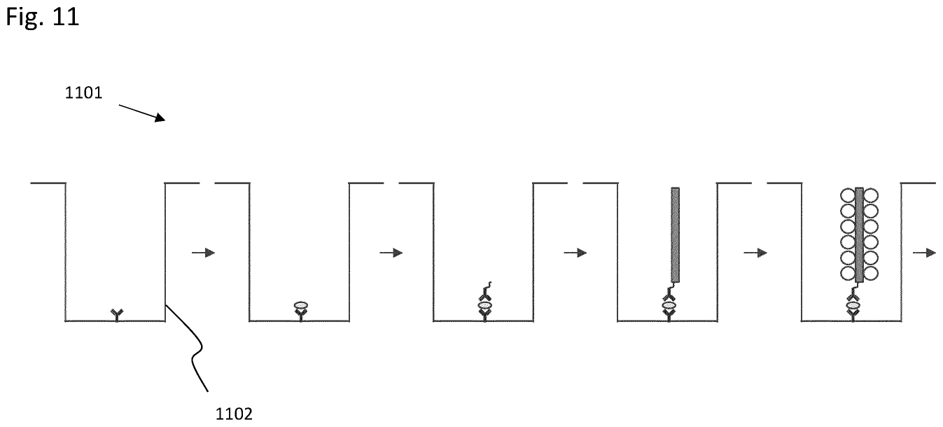

FIG. 11 schematically shows a method of applying the labeling nanostructure according to the invention, in particular the labeling nanostructure formed in FIG. 8a, in an immunoassay format using a well plate.

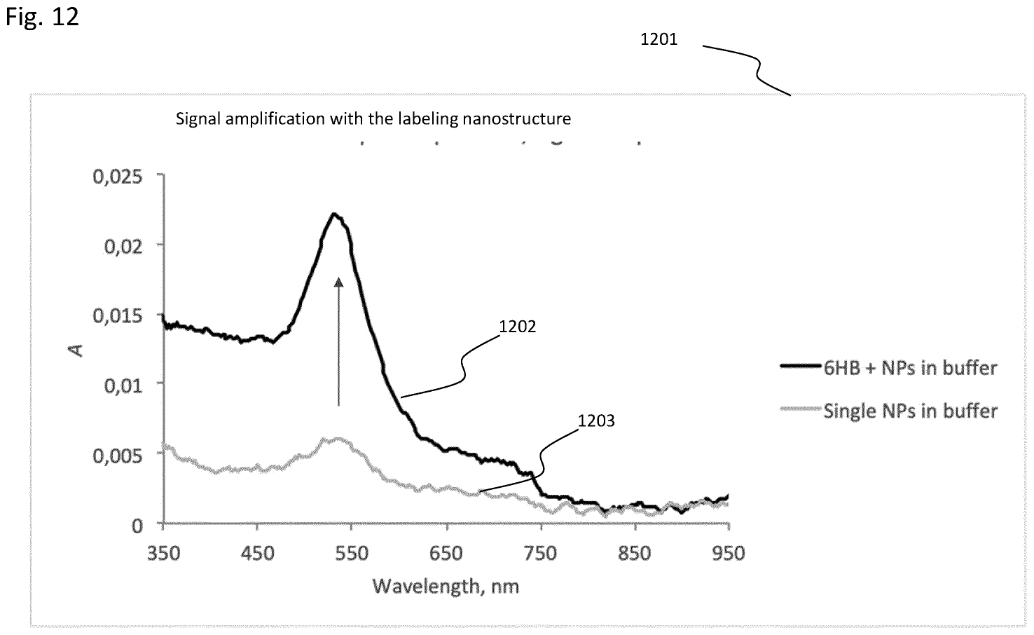

FIG. 12 shows experimental results for demonstrating the signal amplification achieved with the labeling nanostructure produced in FIG. 8a.

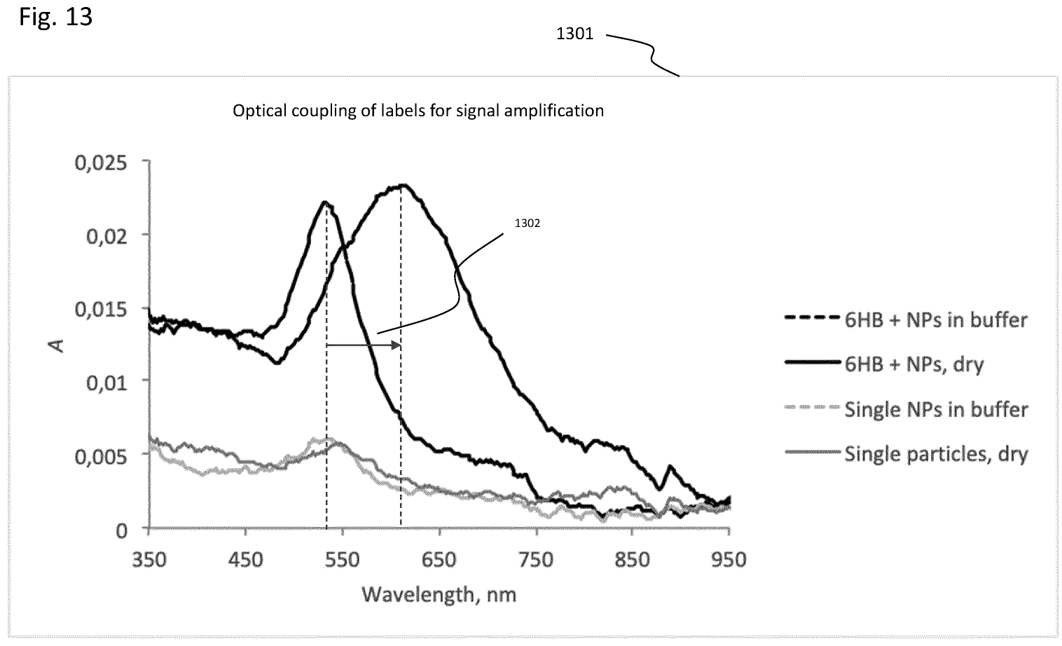

FIG. 13 shows experimental results for demonstrating the signal amplification achieved with the labeling nanostructure produced in FIG. 8a, including the demonstration of coupling and a shift of the plasmon resonance.

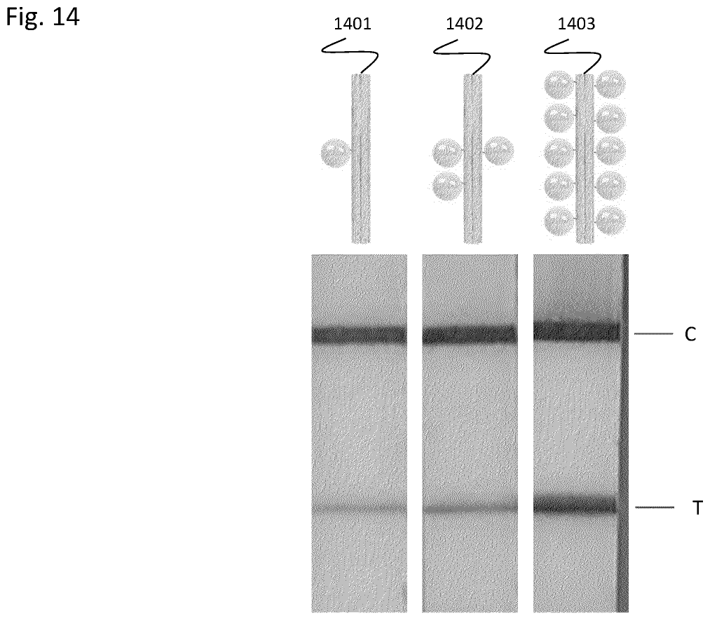

FIG. 14 shows experimental results for demonstrating the signal amplification achieved with the labeling nanostructure produced in FIG. 8a.

FIG. 1a shows an embodiment of a labeling nanostructure 100 labeled with a number of N=6 nanoparticles 107a, which each comprise a functionalized substituent 106. The polynucleotide-based nanostructure 103 is attached via its first binding site 104 to an analyte 101. The first binding site thereby comprises a first binding site sequence 104a, which peeks out of the nanostructure as an anchor strand. To this exposing end of the first binding site 104, a first binding site reagent 104b is attached. The first binding site reagent 104b is selected depending on the analyte 101. The selection is such, that the analyte 101 selectively binds to the binding site reagent 104b and binding to other reagents of the nanostructure 103 is thereby avoided.

In this prescribed embodiment, the analyte 101 comprises two different binding sites. The one binding site of the analyte 101 serves to bind the analyte 101 to the first binding site reagent 104b of the nanostructure 103, whereas the other binding site of the analyte 101 serves to bind the analyte 101 to the target 108.

The target 108 comprises a target molecule 108b and a target binding site 108a. The target binding site 108a serves to bind the target molecule 108b to the analyte 101. The target molecule 108b itself is attached to a substrate 102, e.g., a backing card. Thereby, the target 108 is immobilized. Therefore, when the analyte 101 binds to the target 108, e.g., in an antigen-antibody interaction, the analyte 101 is immobilized. Binding of the analyte 101 to the binding site reagent 104b of the nanostructure 103 further immobilizes the labeling nanostructure 100. In this way, the analyte 101 is immobilized by the target 108 at the position of the target 108 on the substrate 102 and the analyte 101 is labeled by the labeling nanostructure at this position through binding of the analyte 101 to the first binding site 104 of the nanostructure 103.

In a target binding zone (T) of the substrate 102, binding between the target 108 and the analyte 101 and preferably also between the analyte 101 and the first binding site 104 of the nanostructure 103, take place.

In a control binding zone (C) of the substrate 102, the nanostructure 103 is bound to a control 110, which comprises a control binding site 110a and a control molecule 110b. In the embodiment shown in FIG. 1a, the target 108 and control 110 correspond to different antibodies. The target antibodies 108 are bond to the analyte 101 and the control antibodies 110 are bound to the third binding site 109 of the nanostructure 103.

In order to bind the control 110 to the nanostructure 103, the nanostructure 103 comprises a third binding site 109 with a third binding site sequence 109a, which peeks out of the nanostructure 103 as an anchor strand. To this exposing end of the third binding site sequence 109a, a third binding site reagent 109b is attached. The third binding site reagent 109b binds to the control 110 via the control binding site 110a of the control 110. The control molecule 110b is attached to the substrate 102 and is thereby immobilized on the substrate 102. Consequently, also the nanostructure 103 becomes immobilized on the substrate 102 when bound to the control 110.

The binding reactions between the binding site 104 of the labeling nanostructure 100 and the control 110 and/or target 108, respectively take place during the period of time, when the labeling nanostructure 100 is transported through the membrane of the substrate 102.

By immobilizing nanostructures 103 by means of the control 110 to which no analyte 101 is bound, i.e., excess labeling nanostructures 100, the control 110 serves to ensure proper functioning of the test device, e.g., a lateral flow test device. For example, excess antibodies from a conjugate pad of the substrate 102, which have not bound to an analyte 101 and therefore do not immobilize on the T-line, continue to migrate through the substrate 102 until they reach the region of the C line and are immobilized there. A colored T-, C-line, respectively appears. The T-lines indicates an analyte 101 concentration and/or a positive test result, whereas the C-Line indicates a proper functioning of the test device. For example, the membrane of the substrate 102 necessary for the transport of the substances 100; 101 is not clogged.

As a result of immobilizing the labeling nanostructures 100 at the respective C-, T-lines, these lines appear as, e.g., colored C-, T-lines visible by naked eye.

The color of the C-, T-lines results from the detectable labels 107. The detectable labels 107 therefore comprise nanoparticles 107a that provide, for example, a colored appearance of the C-, T-lines due to their optical properties. For example, the nanoparticles 107a may be fluorescent, or they influence the optical perception of the C-, T-lines due to their size and material properties, e.g., as metallic nanoparticles.

As it is further illustrated in FIG. 1a, in order to bind a detectable label 107 to the nanostructure 103, the nanostructure 103 comprises second binding sites 105. The second binding sites 105 are spatially arranged at the nanostructure 103, such that binding of the detectable labels 107 is not spatially hindered through the geometrical dimensions of the nanoparticles 107a, e.g., through their respective radii. This essentially allows each binding site 105 to be occupied by one nanoparticle 107a. Therefore, because the nanostructure 103 allows arranging the detectable labels 107 such that each second binding site 105 can be occupied by one detectable label 107, each of the labeling nanostructures 103 attached to a respective analyte 101 contributes equally to the detection signal of the test device. That is by the same quantity, e.g., the same amount of signal intensity. As the number of second binding sites 105 at each nanostructure 103 is preferably the same, the detection signal of the test device linearly depends on the analyte 101 concentration, which accumulates at the T-line of the substrate 102.

FIG. 1b schematically illustrates a second binding site 105 of the nanostructure 103 and a functionalized substituent 106 of the detectable label 107. A second binding site 105 comprises a binding site sequence 105a, which peeks out of the nanostructure 103 as an anchor strand. To this exposing end of the second binding site sequence 105a, a second binding site reagent 105b is attached. The second binding site reagent 105b binds to the detectable label 107 via the binding reagent 106b of the functionalized substituent 106 of the detectable label 107. In the embodiment as shown in FIG. 1b, the binding reagent of the substituent 106b is bond to the substituent 106a, whereas the substituent 106a is linked to the surface of the nanoparticle 107a via a sulfide bond.

FIG. 2a shows a test device 211 for performing a lateral flow test according to the invention, in a first status of its application. The test device 211 comprises a test substrate 212 as a test strip, made from a porous material, e.g., containing cellulose. The porous material is configured to let a fluid sample 214, for example a medical body liquid, or an aqueous dilution containing the same, flow along a direction F parallel to a length axis of the test strip 212, driven by capillary forces. In a region 213 of the test strip, the detectable labels 107 are deposited as functionalized nanoparticles 107, or in case of multiplexing: different groups of functionalized nanoparticles 107, are located there. The different groups may distinguish by the material properties or sizes of the nanoparticles 107a, in particular by their optical properties. Because of the specific optical properties of the detectable labels 107, they act as visual markers, i.e., to label an analyte 101 for the detection of the analyte 101. In the region 213, the functionalized nanoparticles 107 may already be linked to the nanostructure 103 and the nanostructure may further comprise an antibody connected to its first binding site 104, whereas the antibody is configured to selectively bind to the analyte 101. The test strip further comprises a test line 215 with the immobilized target molecules 108, e.g., antibodies, arranged along the T-line 215, also configured to selectively bind to the analyte 101.

FIG. 2b shows the test device 211 of FIG. 2a, in a second status of its application. The test device 211 is preferably configured to perform a so-called sandwich assay. Sandwich assays may be generally used for larger analytes 101 because they tend to have multiple binding sites. As the fluid sample 214 migrates through the test strip 212 it first encounters in the region 213 a conjugate, which can be the antibody specific to the analyte 101, and which is labelled with the labeling nanostructure 100, and which comprises the functionalized nanoparticles 107. The antibodies bind to the analyte 101 within the sample fluid and migrate together until they reach the test line 215. The test line 215 further contains immobilized antibodies as targets 108 and which are specific to the analyte, i.e., which bind to the analyte, which is connected to the labeling nanostructure 100. The test line 215 then presents a visual change 215′ due to the concentrated visual markers, e.g., the number of nanoparticles 107, thereby confirming the presence of the analyte 101. In case of multiplexing, different groups of different nanoparticles are provided in region 213, and different test lines 215 are located at different positions along the length of the test strip 212.

FIG. 2c shows a diagram describing the method of producing a test device for performing a lateral flow test, including the step 216 of providing a test substrate having a test strip; and the step 217 of applying to the test substrate in a region, e.g., an absorption pad region, a plurality of labeling nanostructures or the alternative step 218 of, applying to the test substrate in the region a plurality of polynucleotide-based nanostructures and a plurality of functionalized nanoparticles as detectable labels, i.e., separately, e.g., as a separate solution. In the case of method step 218, the functionalized nanoparticles can be provided at a position of the absorption pad, which differs from the location were the nanostructures are applied on the absorption pad. The functionalized nanoparticles can be provided after or before application of the nanostructure.

FIG. 3a shows a scheme of a paper-based lateral flow assay 302. The assay 302 comprises an absorption pad 312, which serves to uptake sample solution 314, e.g., containing the analyte 301 as indicated by a drop shaped symbol 314. From the absorption pad 312, which is realized as a cellulose, i.e., paper-based membrane, the number of drops 314 of sample solution applied to the pad 312 are transported by capillary forces towards the next pad region, which comprises the binding zone 308a. The binding zone 308a is pre-treated in that it contains immobilized molecules as targets and as controls. The target molecules sever for immobilizing the analyte 301 with the nanostructure 303 attached to it. The control molecules serve for immobilizing the nanostructure 303 without the analyte 301 being attached to the nanostructure 303. Upon accumulation of several immobilized nanostructures 303 a test line 315 appears on the assay strip, e.g., a colored line, as a result of the optical properties of the detectable labels 306 linked to the nanostructure 303. The detectable labels 306, e.g., functionalized nanoparticles, can be bond to the nanostructure 303 before the nanostructures 303 bind to target and/or control molecules, i.e., before being immobilized. Preferably, the nanostructures 303 first bind to the respective target and/or control molecules and the detectable labels 306 are attached to the respective second binding sites of the nanostructure 303 after the nanostructures 303 are immobilized.

FIG. 3b presents the lateral flow assay of FIG. 3a in more detail. A DNA origami structure 303 is employed to increase the number of nanoparticles 306 that accumulate at a test line 315 on a paper strip 302. Increasing the number of nanoparticles 306 at the test line 315 amplifies the signal used for detecting the analyte 301.

In a first step (1), the nanostructure 303, an about 414 nm long rod with about 7 nm diameter, is made of a 6-helix-bundle (6 HB) with 55 anchor strands 305a (poly A15) presenting second binding sites 305 for the conjugation of nanoparticles 306, which are functionalized with poly T8-25 as functionalized substituents 306b. The 6 HB is immobilized on the strip in the binding zone 308a through the control molecule streptavidin 330. The 6HB nanostructure 303 therefore comprises a biotin molecule as the third binding site reagent 309b. Thus, in the binding zone 308a the streptavidin—biotin 330/309b complex forms and immobilizes the 6HB nanostructure 303.

The 6HB nanostructure 303 is therefore mixed in excess with the biotin-DNA to allow hybridization. The mixture 318 is transferred and allowed to migrate through the LFA strip 302, which has streptavidin 110 pre-immobilized in the binding zone 308a. Once the 6 HB-biotin conjugate encounters the streptavidin 110 it binds via streptavidin-biotin interaction.

A washing step is carried out using buffer to flush excess and unbound 6 HBs from the membrane 302.

In a last step (2), functionalized nanoparticles 306 are allowed to migrate through the membrane 302 and are captured by the 6 HB via DNA hybridization of poly A and poly T strands.

In the preferred embodiment as shown in FIGS. 3a, 3b the lateral flow assay (LFA) uses a DNA origami. Therefore, in the first step (1) as indicated in FIG. 3b, a buffer solution containing a final concentration of 20 mM Tris, 100 mM NaCl, 0.1% SDS, and 1 mg/ml BSA with pH 8 is prepared. To 5.5 L of that solution, 3 L of 0.1 M MgCl₂ and 20 μL of 2.5 nM 6 HB (in 1×TAE 11 mM MgCl₂) is added. Then, 1.5 μL of 10, 5, 2.5, 1, 0.5 pM biotin-DNA is added resulting in final concentrations of 250, 125, 75, 50, 25 fM. The final MgCl₂ concentration in each sample is 10 mM. In step (2) as further indicated in FIG. 3b, the mixture is transferred to the absorption pad 312 of the LFA strip 302. After a few minutes the whole liquid is migrated through the membrane 302 and soaked up by the waste pad 311. Then, 40 L of the initially prepared buffer is allowed to flow through the strip 302 washing off residuals from the membrane. In a last step (not shown in FIG. 3b), 10 μL of 1 nM polyT functionalized gold nanoparticles 306 are added to the absorption pad 312. After a few minutes, a colored line 315 appears on the LFA strip indicating the test result.

FIG. 3c shows the different sizes of spherical and rod-shaped functionalized nanoparticles 306 that can be employed, and which are captured by the 6 HB nanostructure 303. To confirm the hybridization process on the strip 302, the 6 HBs and nanoparticles 306 are added together and immediately transferred to a transmission electron microscope (TEM) grid for analysis. The DNA origami-nanoparticle-assemblies 300 indicate a very fast hybridization process. As this process is carried out on a LFA its kinetics is an important factor effecting the quality of the assay. The use of DNA origami nanostructure 303 to capture more nanoparticles 306 that would usually accumulate on the membrane 302 gives rise to a very high color intensity of the test line 315. This strategy enables an increase in sensitivity.

FIG. 4 presents substrates 402 of several lateral flow immunoassays after an analyte concentration has been detected, which is indicated by the respective C-lines 420 and T-lines 415 as shown in FIG. 4. The analyte concentration varied for each test by a factor of 10. Starting from an analyte concentration of 1 nano mole (nM) in a first test assay, the analyte concentration was diluted for each test. In a last test assay an analyte concentration of 1 femto mole (fM) was detected. The C-lines 420 visibly appear for all the different analyte concentrations as expected. As expected, the appearance of the T-lines 415 becomes weaker with decreasing analyte concentration, as the measurement signal is amplified by fewer and fewer immobilized labeling nanostructures. However, since the number of detectable labels per nanostructure and thus per analyte molecule is high, e.g., 55 functionalized nanoparticles per nanostructure, even a low analyte concentration of 1 pM can be detected as it appears from the T-lines 415 shown in FIG. 4. The T-line 415 in the case of the further reduced analyte concentration of 100 fM is also still visible to the eye, although less contrasted. As analyte the Sars-COV 2 virus was selected, whereas Digoxigenin served as control molecule. Amplification through the labeling nanostructure allows detection of a Sars-COV 2 concentration of up to 100 fM by eye, which is 1 pg/mL, corresponding to 8000 g/mol DNA sequence.

FIG. 5 shows T-lines 515a, 515b of pairs of substrates at different analyte concentrations. Starting at an analyte concentration of 10 nM for a first pair of substrates, the concentration of the analyte decreases by a factor of 10 for each additional pair of substrates shown in FIG. 5, down to 10 fM for the last pair of substrates. As expected, the intensity of the T-lines 515a, 515b on the substrates for each pair decrease with decreasing analyte concentration. The decrease in the intensity of the measured signal is plotted by the height of an intensity bar shown above each of the substrate pairs. The substrate pairs differ by the amplification of the measurement signal. For the top row of substrates 515a shown in FIG. 5, a single 40 nm gold particle is used to detect a respective single analyte molecule. For the bottom row of substrates 515b shown in FIG. 5, the labeling nanostructure with, e.g., 55 nanoparticles are used to detect a respective single analyte molecule, thereby amplifying the measurement signal. Therefore, in case of the conventional LFA using 40 nm gold labels, an analyte concentration below 100 pM (pico Mole) cannot be detected by eye as the intensity drops below visibility of the human eye. For example, the intensity bar for 10 pM reaches almost zero intensity. The detection limit is indicated by the dotted line in the intensity diagram of FIG. 5. However, amplification of the measurement signal means the detectable nanostructures as claimed allows reading the test lines up to an analyte concentration of 100 fM with the intensity bar being similar in height to the one for a concentration of 100 pM in case of 40 nm gold particle labels.

FIG. 6 shows an embodiment of the method for providing signal amplification to an immunoassay, which uses a single detectable label 606. The single detectable label as shown is bound to an antibody 604, which further binds to the analyte 601, which is immobilized on the substrate 602 through binding to the antibody 605. The method is based on the concept to substitute the conventionally used single label 606 (1), with a labeling nanostructure 600 (2). Therefore, the conventionally used single label 606 is substituted in a first step by a nanostructure 603, configured to bind a predetermined number of detectable labels 606. In a second step, a multitude of, e.g., ten detectable labels 606 as illustrated in FIG. 6, e.g., functionalized nanoparticles, are attached to the nanostructure 603, thereby increasing the signal intensity compared to a single detectable label about ten times. Alternatively, the nanostructure 603 can be equipped with the detectable labels 606 in a first step, before substitution. In FIG. 6 also a TEM 608 image is provided as an inset. The TEM image 608 shows the labeling nanostructure 600 with the detectable labels 606 attached. The respective test line 607 of the substrate 602 is shown in FIG. 6, whereas the lower intensity, i.e., the lower amplification of the measurement signal is indicated by the lower brightness of the respective test line 607.

FIG. 7 shows an embodiment where the labeling nanostructure 700 is linked to a bead 701, as it is typically the case in bead-based immunoassays. Therefore, the bead surface is modified, i.e. coated with, for example antibodies 702, which selectively bind to an analyte 703. The analyte further binds to another detection antibody 704, which is bond to the first binding site of the nanostructure 705. In the embodiment shown in FIG. 7, the second binding sites of the nanostructure 705 each comprise a detectable label 706. Therefore, the labeling nanostructure 700 amplifies the detection signal by a factor of 10, as there are 10 detectable labels 706 bound to the nanostructure 705 compared to a conventional single label detection mechanism, e.g. a fluorescent nanoparticle 706 is directly bound to the detection antibody 704.

FIG. 8a shows a method of producing an inventive labeling nanostructure, according to an embodiment of the invention. FIG. 8b shows an embodiment of applying the labeling nanostructure in an immunoassay, the labeling nanostructure containing detectable labels, here polynucleotide-modified labels, according to an embodiment of the invention, which labeling nanostructure may be produced, in particular, using the method explained by FIG. 8a, and which labeling nanostructure is bound to an analyte 809 coupled to a substrate 811.

The labeling nanostructure according to the embodiments of the invention for signal amplification is used in different immunoassay formats e.g. sandwich immunoassays, competitive immunoassays or other formats, well known to those with skill in the art. FIG. 8 schematically illustrates the application of the labeling nanostructure on a lateral flow sandwich immunoassay for the detection of cardiac troponin I. The polynucleotide-based nanostructure (generally also referred to as “polynucleotide nanostructure”) forms from a polynucleotide scaffold 801, provided in step P1, and a set of polynucleotide staples 802.

The polynucleotide sequences of the staples and the scaffold predefine the shape and the size of the final polynucleotide nanostructure 803, provided in step P2. For high signal amplification a large number of second binding sites with large binding-site distances are included. In this case an elongated filament with 428 nm length and 8 nm diameter is used. A modified antibody (e.g. a polynucleotide modified cardiac troponin I antibody) is conjugated to the polynucleotide nanostructure 804, in step P3. The polynucleotide linker is attached to the antibody via copper-free click-chemistry (reaction of a diarylcyclooctyne moiety (DBCO) with an azide-modified polynucleotide reaction partner). The polynucleotide nanostructure 805 with the antibody attached to the first binding site is formed in step P4. Addition of suitably modified labels 806 in step P5 leads to the fully assembled labeling nanostructure 807, provided in step P6.

The labeling nanostructure can be applied in various immunoassay formats, shown exemplarily in step P7 of FIG. 8b, e.g. sandwich immunoassays 808. The antibody pair 804 and 810 is adapted to various analytes 809 by those with skill in the art. Signal amplification is tailored by those with skill in the art by adaptation of the polynucleotide nanostructure with tailored numbers of first and second binding sites. The substrate 811 may be a porous membrane in case of a lateral flow immunoassay, or a bead in case of a bead based immunoassay, or generally a binding area on a substrate (microfluidic channel, well-plate).

FIG. 9a shows experimental results from gel electrophoresis for demonstrating the synthesis of the polynucleotide nanostructure formed in FIG. 8a. The defined size and molecular weight of the polynucleotide nanostructure is confirmed by agarose gel electrophoresis (901-904). A molecular ruler is shown in 902, the polynucleotide scaffold in 903, the polynucleotide nanostructure with precise, pre-defined size and molecular weight and excess staples in 904. A distinct band is observed that represents the folded polynucleotide nanostructures.

FIG. 9b shows experimental results from transmission electron micrography for demonstrating the synthesis of the polynucleotide nanostructure formed in FIG. 8a. The picture is a transmission electron micrograph 905 of the elongated filament polynucleotide nanostructure.

FIG. 10 shows the application of the polynucleotide nanostructure formed in FIG. 8a, in a lateral flow immunoassay in sandwich format. Signal amplification on a cardiac troponin I lateral flow immunoassay in sandwich format. 1001 shows results for troponin detection with colloidal gold based lateral flow assay without signal amplification; the picture is a photograph of the porous membrane, on which the LFA was performed, in a top view. In the given sample matrix (buffer system) the concentration of 1 nM troponin I is not detectable (1002). 1003 shows results for successful detection of 1 nM troponin I after signal amplification with the labeling nanostructure (1004).

FIG. 11 schematically shows a method of applying the labeling nanostructure according to the invention, in particular the labeling nanostructure formed in FIG. 8a, in an immunoassay format using a well plate. The polynucleotide nanostructure can be applied in various immunoassay formats. Shown here (1101) is the integration of the polynucleotide nanostructure in a sequential immunoassay protocol on the surface of a well 1102 of microfluidic wells or 96-well/384-well plate, for example. Signal is read out with a spectrometer, in this case a plate reader (fluorescence or UV-VIS absorption depending on the label).

FIG. 12 shows experimental results for demonstrating the signal amplification achieved with the labeling nanostructure produced in FIG. 8a. Shown are results of a cardiac troponin I sandwich immunoassay with/without signal amplification performed in a 384-well plate format (1201). The analyte concentration is 125 picomoles per liter. The signal is read out via UV-VIS spectroscopy in a plate reader. Conventional antibody conjugates without signal amplification lead to low signal intensity (1202). Labeling nanostructures amplify the signal (1203) significantly. The magnitude of signal amplification can be tailored by adjusting the number of second binding sites on the polynucleotide nanostructure.

FIG. 13 shows experimental results for demonstrating the signal amplification achieved with the labeling nanostructure produced in FIG. 8a, including the demonstration of coupling and a shift of the plasmon resonance. UV-VIS readout of a sandwich immunoassay after signal amplification (1301). The polynucleotide nanostructure leads to controlled assembly of multiple colloidal labels in close proximity. In this case, 40 nm gold nanoparticles are brought in close proximity with a surface-to-surface distance of only a few nanometers (1-3 nm). The close distance leads to coupling and a shift of the plasmon resonance. Shown here is the experimental UV-VIS measurement of a redshift of around 80 nanometers (1302) after signal amplification in a cardiac troponin I immunoassay (384-well plate format). The particles get in closest distance when the assay plate is dried. Individual/single nanoparticles do not show significant resonance shifts. Resonance shifts due to optical coupling of labels provide an approach to stronger signal amplification with the polynucleotide based labeling nanostructures.

FIG. 14 shows experimental results for demonstrating the signal amplification achieved with the labeling nanostructure produced in FIG. 8a. Varying the number of second binding sites leads to predefined signal values. Shown here are three lateral flow assays of polynucleotide nanostructures with different numbers of second binding sites. The three pictures show, respectively, a photograph of the porous membrane, on which the LFA was performed, in a top view. The number of second binding sites increases from left to right (1401-1403). The increasing number of second binding sites results in a stronger signal at the target binding area.

Detailed description of the polynucleotide nanostructure, used in particular in FIGS. 8 to 14:

The geometry of the polynucleotide-based nanostructure is preferably designed to maximize the number of bound detectable labels, in particular polynucleotide-modified labels, and thus maximize signal amplification. High aspect ratios of the polynucleotide-based nanostructures enable dense populations of polynucleotide-based nanostructures on surfaces. In embodiments, in particular in FIGS. 8 to 14, the polynucleotide-based nanostructure is designed as an elongated, high aspect-ratio filament of, in particular, 428 nm length and 8 nm diameter. It features a total of 172 second binding sites along its elongation axis. Based on geometrical considerations, 10 layers of 3 polynucleotide-modified labels per layer—equalling 30 polynucleotide-modified labels in total—can be attached. Each attached polynucleotide-modified label binds to several second binding sites to maximize binding stability. One to six polynucleotide-modified detection antibodies are bound to each end of the polynucleotide nanostructure. The polynucleotide-modified labels are attached in a geometry that prevents unwanted steric hindrance of detection-antibody/analyte interaction from the relatively large polynucleotide-modified labels. If necessary (e.g. to modify reaction kinetics of analyte binding), polynucleotide-modified detection antibodies can also be attached to designated positions along the elongation axis of the polynucleotide nanostructure. Stronger signal amplification is achieved by connecting multiple polynucleotide nanostructure monomers into a multimer via specific interactions of the ends of the polynucleotide nanostructures.

If necessary, the overall shape and size of the polynucleotide nanostructure can be designed to fit the needs of specific immunoassay reaction systems, e.g. to modify transport kinetics via rationally engineering the translational diffusion coefficients for axial and transverse movement in microfluidic channels or nitrocellulose membranes with smaller pore sizes.

The first and second binding sites expose single stranded polynucleotides. From our experimental data, the following sequences are preferred:

TABLE 1
Sequences for first and second binding sites

| Name | Sequence (5′→3′) | Modification |
|---|---|---|
| First binding site 1 | AAAGAGGAGAGAAGAGAAGGAAGGA-staple | |
| First binding site 2 | GGAGAGAGAAGGAAGAGGAAAGAAG- | |
| | staple | |
| Anti-first binding | TCCTTCCTTCTCTTCTCTCCTCTTT-Azid | 3′ Azid |
| site 1 (antibody 1) | | |
| Anti-first binding | CTTCTTTCCTCTTCCTTCTCTCTCC-Azid | 3′ Azid |
| site 2 (antibody 2) | | |
| Label | Thiol-TTTTT TTTTT TTTTT TTTT | 5′ Thiol |
| (nanoparticle) | | |
| Second binding | staple-AAAAA AAAAA AAAAA AAAAA | |
| site | | |

Embodiments of the invention, in particular the embodiments of FIGS. 8 to 14, may be implemented using any or all of the following conditions, which may encompass materials, methods, regarding the polynucleotide-based nanostructure, polynucleotide-modified detection antibodies (detectable labels), polynucleotide-modified colloidal gold labels, colloidal-gold conjugates, the lateral flow immunoassay, the plate-based sandwich immunoassay, all being composed according to aspects of the present invention:

### Materials+Methods

### Polynucleotide-Based Nanostructure

### Materials:

Polynucleotide scaffold (M13mp18 based, nucleotide length N preferably between 7249<N<8634)
Polynucleotide staples, pooled
Folding buffer (e.g. 1× Tris-EDTA buffer, pH 7.5 containing 20 mM MgCl₂ and 5 mM NaCl)
Storage buffer (e.g. 1× Tris-EDTA buffer, pH 7.5 containing 5 mM MgCl₂ and 5 mM NaCl)

### Methods:

The polynucleotide nanostructures are prepared as follows: the polynucleotide scaffold is mixed with the pooled polynucleotide staples in molar excess (preferably between 5 and 100 times molar excess) and folding buffer. The mixture is heated to 65° C. for 15 min and subsequently cooled down to 25° C. over the course of preferably 1 hour to 16 hours. The assembled polynucleotide nanostructures are subsequently purified from the excess of polynucleotide staples via size exclusion HPLC. After purification, the buffer is exchanged and the concentration adjusted to the desired value (between 1 to 1000 nmole/liter) via ethanol precipitation or spin filtration.

### Polynucleotide-Modified Detection Antibodies

### Materials:

Monoclonal IgG detection antibody specific to the target analyte
DBCO-sulfo-NHS ester crosslinker
Azide-modified polynucleotides
Reaction buffer, e.g. phosphate-buffered saline (PBS) at pH 7.4
Centrifugal filters with a 10 kDa and a 100 kDa molecular weight cutoff
Desalting spin columns with a 40 kDa molecular weight cutoff

### Methods:

The detection antibodies are covalently conjugated to polynucleotides through a DBCO-sulfo-NHS ester crosslinker. Covalent conjugates are formed when first the surface lysines (primary amines) of the IgG molecule react with the NHS ester moiety of the crosslinker, and subsequently the DBCO groups react with the azide groups on the polynucleotides. Depending on the identity of the detection antibody, other covalent or non-covalent conjugation methods may be preferred.

If necessary, a buffer exchange of the labeled IgG detection antibody to the reaction buffer is first carried out by spinning with 10 kDa molecular weight cutoff spin-filters according to the manufacturer's guidelines. The crosslinker is mixed with the antibody in a preferably 5-30-fold molar excess of crosslinker to antibody in the reaction buffer, and incubated at either room temperature for 30 minutes or at +4° C. for 2-3 hours. The unreacted cross linker molecules are removed from the sample using a desalting spin column. Azide-modified polynucleotides are mixed with the antibodies in the reaction buffer in a 5-20-fold molar excess of polynucleotides to protein and incubated typically overnight at +4° C. The reaction mixtures are purified from excess azide-polynucleotides by spinning with 100 kDa molecular weight cutoff spin-filters according to the manufacturer's guidelines.

### Polynucleotide-Modified Colloidal Gold Labels

### Materials:

Colloidal gold nanoparticles of desired diameter, preferably 40 nm
Thiol-modified polynucleotides
Washing buffer, e.g. 0.02% SDS in MQ-water

### Methods:

Gold nanoparticles are mixed with a molar excess of thiolated DNA and frozen at −20° C. for 5 h. After thawing, samples are washed by iterative centrifugation (5×) and resuspended in 0.02% SDS. In the last step, the pellet is resuspended in a small volume of 0.02% SDS to concentrate the particles. The concentration is determined by UV-VIS spectroscopy at 520 nm.

### Colloidal-Gold Conjugates

### Materials:

Colloidal gold nanoparticles of desired diameter, preferably 40 nm
Monoclonal IgG detection antibody specific to the target analyte
Modification buffer, e.g. Tris-HCl buffer (pH 6.5)

### Methods:

Gold nanoparticles are centrifuged and the pellet is resuspended in Tris-HCl buffer (pH 6.5). The monoclonal IgG detection antibody specific to the target analyte is added in molar excess to the nanoparticles. After incubation for 1 hr, the particles are washed by iterative centrifugation (5×) and resuspended in 1xPBS.

### Lateral Flow Immunoassay

### Materials:

Lateral flow immunoassay strips with capture antibodies blotted on the target line Polynucleotide-modified detection antibodies, polynucleotide nanostructures, polynucleotide-modified labels, and colloidal-gold conjugates prepared as described earlier. The polynucleotide-modified detection antibodies, labels, and the polynucleotide nanostructure are diluted to a concentration c of preferably 100 pmol/L<c<100 nmol/L in either the blocking buffer or other application- or analyte-specific diluent.

Buffer, serum, or blood sample containing the target analyte.

Blocking buffer for passivating the nitrocellulose membrane; e.g. PBS supplemented with blocking agents such as bovine serum albumin (BSA), casein, whole serum, Tween-20, or similar.

### Methods:

The buffer, serum, or blood sample containing the target analyte is mixed with the signal amplification solution containing the polynucleotide-modified detection antibodies and polynucleotide nanostructures. The mixture is pre-incubated (typically between 1 min<t<30 min) and the analyte concentration subsequently detected on the lateral flow immunoassay strips with the polynucleotide-modified labels.

### Plate-Based Sandwich Immunoassay

### Materials:

Microwell plates with a high protein-binding capacity (e.g. Thermo Scientific Nunc Maxisorp)

Coating buffer suitable for the immobilization of the capture antibody through passive adsorption, e.g. sodium bicarbonate buffer at >pH 9.0.

Blocking buffer for passivating the plate surface after the immobilization of the capture antibody; e.g. PBS supplemented with blocking agents such as bovine serum albumin (BSA), casein, whole sera, Tween 20, or similar.

Wash Buffer, Such as PBS or TBS with Tween 20

The monoclonal IgG capture antibody, diluted in the coating buffer.

Polynucleotide-modified detection antibodies, polynucleotide nanostructures, and polynucleotide-modified labels, prepared as described earlier. The polynucleotide-modified detection antibodies, labels, and the polynucleotide nanostructure are diluted to a concentration c of preferably 100 pmol/L<<<100 nmol/L in either the blocking buffer or other application- or analyte-specific diluent.

Buffer, serum, or blood sample containing the target analyte.

### Methods:

Assay plates are prepared by immobilizing the capture antibody on the plate surface through the selected coupling method. For passive adsorption, the capture antibody is diluted in the coating buffer and incubated in the assay wells typically overnight at +4° C. The wells are washed with the wash buffer and incubated with the blocking buffer, typically 2-3 hours at room temperature or at +37° C., to passivate the surface with the blocking agent(s). The wells are washed with the wash buffer and the sample containing the target analyte is added. The incubation time and temperature for the sample on the plate, as well as for the subsequent incubations with the other assay components, are optimized for each assay setting. For a sequential assay, the wells are then washed, incubated with the polynucleotide-labeled detection antibodies, washed, incubated with the polynucleotide nanostructures, washed, incubated with the polynucleotide-modified labels, and finally washed to remove all unbound labels. Depending on the type of label used, a plate reader based read-out of either the absorbance, fluorescence, chemiluminescence, or other optical property of the labels is then used to quantify the amount of analyte in the studied sample. The measurement can be performed either after filling the wells with a buffer of choice, or from dry wells.

For the following human health applications, an in vitro diagnostic solution is available in the form of a laboratory test or a point-of-care test. According to the invention, a lateral flow assay can be developed for those applications. Classification is according to the ICD (Classification of diseases) of the World Health Organization. Therefore, the hereby provided list of applications relates to corresponding analytes of each respective disease.

### A. Infectious Agents

Virus: Coronavirus (Sars-cov-2); Hepatitis virus; Human herpesvirus; Human papillomavirus; Influenza virus; Epstein-Barr virus; Cytomegalovirus; Zika virus; Human respiratory syncytial virus
Bacteria: Chlamydia; Gonorrhoeae; Streptococcus.

### B. Human Diseases and Disorders

Infectious or parasitic diseases: Sepsis; Trichomoniasis; Viral hepatitis; Meningococcal disease; Tuberculosis; Chagas; disease; Syphilis; HIV; Ebola disease; Chikungunya virus disease; Dengue.

Neoplasms: Prostate cancer; Liver cancer; Colorectal cancer; Cervical cancer; Breast cancer; Bladder cancer; Lung cancer.

Diseases of the blood or blood-forming organs: Leukemia; Megaloblastic anaemia due to vitamin B12 deficiency; Pernicious anemia Diseases of the immune system

Connective tissue diseases; Antiphospholipid syndrome; Antineutrophil cytoplasmic antibody-associated vasculitis; Immunodeficiency.

### Allergic or Hypersensitive Conditions:

Abalone allergy; Acacia allergy; Acremonium kiliense (Cephalosporium acremonium) allergy; Alfalfa allergy; Almond allergy; Alternaria alternata allergy; American beech allergy; American Cockroach allergy; Amoxicilloyl allergy; Ampicilloyl allergy; Anchovy allergy; Animal proteins allergy; Anisakis allergy; Anise allergy; Apple allergy; Apricot allergy; Arizona cypress allergy; Ascaris allergy; Asparagus allergy; Aspergillus flavus allergy; Aspergillus fumigatus allergy; Aspergillus niger allergy; Aspergillus oryzae allergy; Aspergillus terreus allergy; Aspergillus Versicolor allergy; Asthma allergy; Aubergine, eggplant allergy; Aureobasidium pullulans allergy; Australian pine allergy; Avocado allergy; Bahia grass allergy; Bald cypress allergy; Bamboo shoot allergy; Banana allergy; Barley allergy; Basil allergy; Bay leaf allergy; Bayberry allergy; Beef allergy; Berlin beetle allergy; Bermuda grass allergy; Birch allergy; Black pepper allergy; Blackberry allergy; Blood worm allergy; Blue mussel allergy; Blue vetch allergy; Blueberry allergy; Botrytis cinerea allergy; Box-elder allergy; Brazil nut allergy; Broccoli allergy; Brome grass allergy; Bromelain allergy; Brussel sprouts allergy; Buckwheat allergy Budgerigar droppings allergy; Budgerigar feathers allergy; Bumble bee allergy; Cabbage allergy; Cacao allergy; Cagebirds allergy; Camomile allergy; Canary bird droppings allergy; Canary bird feathers allergy; Canary grass allergy; Candida albicans allergy; Caraway allergy; Cardamon allergy; Careless weed allergy; Carob allergy; Carp allergy; Carrot allergy; Cashew nut allergy; Cat allergy; Cat dander allergy; Catfish allergy; Cauliflower allergy; Cedar allergy; Cedar elm allergy; Cefaclor allergy; Celery allergy; Chaetomium globosum allergy; Cheese, cheddar type allergy; Cherry allergy; Chestnut allergy; Chick pea allergy; Chicken allergy; Chicken droppings allergy; Chicken feathers allergy; Chicken serum proteins allergy; Chilipepper allergy; Chinchilla epithelium allergy; Chlorhexidine allergy; Chub mackerel allergy; Cladosporium Cladosporioides allergy; Cladosporium herbarum allergy; Clam allergy; Clove allergy; Cocklebur allergy; Cockroach, German allergy; Cocksfoot allergy; Coconut allergy; Cod allergy; Coffee allergy; Common millet allergy; Common pigweed allergy; Common ragweed allergy; Common reed allergy; Common silver birch allergy; Common wasp allergy; Common wasp venom (Yellow jacket) allergy; Coriander allergy; Cotton seed allergy; Cotton, crude fibers allergy; Cottonwood allergy; Cow allergy; Cow dander allergy; Cow's milk whey allergy; Crab allergy; Crayfish allergy; Cucumber allergy; Cultivated oat allergy; Cultivated rye allergy; Cultivated wheat allergy; Curry allergy; Curvularia lunata allergy; Cypress allergy; Dandelion allergy; Date allergy; Dill allergy; Dog allergy; Dog dander allergy; Dog fennel allergy; Douglas fir allergy; Duck feathers allergy; Eel allergy; Egg allergy; Egg white allergy; Egg yolk allergy; Elder allergy; Elk/moose meat allergy; Elm allergy; Epicoccum purpurascens allergy; Ethylene oxide allergy; Eucalyptus, Gum-tree allergy; European ash allergy; European hornet venom allergy; European Paper wasp allergy; European paper wasp venom allergy; False ragweed allergy; Feathers allergy; Fennel, fresh allergy; Ferret epithelium allergy; Ficus spp. Allergy; Fig allergy; Finch feathers allergy; Fire ant allergy; Firebush (Kochia) allergy; Fish allergy; Fish (cod) allergy; Food allergy; Formaldehyde/Formalin allergy; Foxtail millet allergy; Fruits allergy; Fusarium proliferatum allergy; Gal allergy; Garlic allergy; Gelatin bovine allergy; Giant ragweed allergy; Ginger allergy; Gluten (Wheat) allergy; Goat epithelium allergy; Goat milk allergy; Goldenrod allergy; Goose feathers allergy; Goosefoot, Lamb's quarters allergy; Grape allergy; Grapefruit allergy; Grass pollen allergy; Green bean allergy; Green coffee bean allergy; Green pepper (unripe seed) allergy; Grey alder allergy; Grouper allergy; Guar, guar gum allergy; Guinea pig epithelium allergy; Gulf flounder allergy; Gum arabic allergy; Hackberry allergy; Haddock allergy; Hake allergy; Halibut allergy; Hamster epithelium allergy; Hazel allergy; Hazelnut allergy; Hemp allergy; Herring allergy; Hexalhydrophtalic anhydrid allergy; Honey allergy; Honey bee allergy; Honey bee venom allergy; Hop allergy; Horn beam allergy; Horse allergy; Horse chestnut allergy; Horse dander allergy; Horse fly allergy; House dust allergy; House dust (Greer Labs. Inc.) allergy; House dust (Hollister-Stier Labs.) allergy; House dust mite allergy; Inhalativer Allergie Test allergy; Insulin human allergy; Isocyanate HDI allergy; Isocyanate MDI allergy; Isocyanate TDI allergy; Ispaghula allergy; Italian/Mediterranean/Funeral cypress allergy; Jack mackerel, Scad allergy; Japanese cedar allergy; Japanese Hop allergy; Johnson grass allergy; Kiwi allergy; Kiwi fruit allergy; Langust (spiny lobster) allergy; Latex allergy; Lemon allergy; Lentil allergy; Lettuce allergy; Lima bean allergy; Lime allergy; Linden allergy; Lobster allergy; London plane tree allergy; Lovage allergy; Lupin allergy; Lupine seed allergy; Macadamia nut allergy; Mackerel allergy; MAHI MAHI allergy; Maize, Corn allergy; Malassezia spp. Allergy; Malt allergy; Mango allergy; Maple leaf sycamore, London plane allergy; Mare's milk allergy; Marguerite, Ox-eye daisy allergy; Marjoram allergy; Meadow fescue allergy; Meadow foxtail allergy; Meadow grass, Kentucky blue allergy; Meat allergy; Mediterranean flour moth allergy; Megrim allergy; Melaleuca, Cajeput-tree allergy; Melon allergy; Mesquite allergy; Milk allergy; Milk boiled allergy; Mink epithelium llergy; Morphine allergy; Mosquito allergy; Moth allergy; Moulds llergy; Mountain juniper allergy; Mouse epithelium allergy; Mouse erum proteins allergy; Mouse urine proteins allergy; Mucor acemosus allergy; Mugwort allergy; Mulberry allergy; Mushroom champignon) allergy; Mustard allergy; Mutton allergy; Nettle llergy; Nuts allergy; Oak allergy; Oat allergy; Octopus allergy; Oil alm allergy; Olive allergy; Onion allergy; Orange allergy; Orange oughy allergy; Oregano allergy; Oyster allergy; Pacific squid llergy; Pancreatin allergy; Papaya allergy; Paper wasp venom llergy; Paprika, Sweet pepper allergy; Parakeet droppings allergy; Parrot feathers allergy; Parsley allergy; Passion fruit allergy; Pea llergy; Peach allergy; Peanut allergy; Pear allergy; Pecan nut llergy; Pecan, Hickory allergy; Penicillin allergy; Penicillium allergy; Penicillium chrysogenum allergy; Penicillium glabrum allergy; Penicilloyl G allergy; Penicilloyl V allergy; Peppertree allergy; Peroxidase allergy; Persimon (kaki fruit, sharon) allergy; Pholcodine allergy; Phoma betae allergy; Phthalic anhydride llergy; Pigeon feathers allergy; Pine allergy; Pine nut, pignoles llergy; Pineapple allergy; Pistachio allergy; Plaice allergy; Plantain llergy; Plantain (English), Ribwort allergy; Plum allergy; Pollock llergy; Poppy seed allergy; Pork allergy; Potato allergy; Privet llergy; Pumpkin allergy; Queen palm allergy; Quinoa allergy; Rabbit allergy; Rabbit epithelium allergy; Rabbit serum proteins llergy; Rabbit, urine proteins allergy; Ragweed allergy; Rape llergy; Rape seed allergy; Raspberry allergy; Rat epithelium llergy; Rat epithelium, serum and urine proteins allergy; Rat serum roteins allergy; Rat urine proteins allergy; rBet v 4, Birch allergy; Red cedar allergy; Red currant allergy; Red kidney bean allergy; Red mulberry allergy; Red snapper allergy; Redtop, Bentgrass llergy; Regional allergy; Regional mix allergy; Rhizopus nigricans llergy; Rice allergy; Rocuronium allergy; Rodents allergy; Rough marshelder allergy; Rye allergy; Rye-grass allergy; Salmon allergy; Saltwort allergy; Saltwort (prickly), Russian thistle allergy; Sardine Pilchard) allergy; Scale, Lenscale allergy; Scallop allergy; Scotch room allergy; Seminal fluid allergy; Sesame seed allergy; Setomelanomma rostrata (Helminthosporium halodes) allergy; Sheep epithelium allergy; Sheep milk allergy; Sheep sorrel allergy; Sheep whey allergy; Shrimp allergy; Silk allergy; Silk waste llergy; Snail allergy; Sole allergy; Soy allergy; Soybean allergy; Spelt heat allergy; Spices allergy; Spinach allergy; Spruce allergy; Squid llergy; Staphylococcal enterotoxin A allergy; Staphylococcal nterotoxin B allergy; Staphylococcal enterotoxin C allergy; Stemphylium herbarum allergy; Storage mite allergy; Strawberry llergy; Streptavidin allergy; Sugar-beet seed allergy; Sunflower llergy; Sunflower seed allergy; Suxamethonium (Succinylcholine) llergy; Sweet chestnut allergy; Sweet gum allergy; Sweet potato llergy; Sweet vernal grass allergy; Swine allergy; Swine epithelium llergy; Swine, urine proteins allergy; Swordfish allergy; Tarragon llergy; Tea allergy; Thyme allergy; Tilapia allergy; Timothy allergy; Tomato allergy; Tragacanth allergy; Tree pollen allergy; Tribolium onfusum allergy; Trichoderma viride allergy; Trichophyton rubrum llergy; Trimellitic anhydride, TMA allergy; Trout allergy; Tuna llergy; Turkey feathers allergy; Turkey meat allergy; Vanilla allergy; Vegetables allergy; Velvet grass allergy; Virginia live oak allergy; Wall pellitory allergy; Walleye pike allergy; Walnut allergy; Watermelon allergy; Weed pollen allergy; Weeds allergy; Western agweed allergy; Wheat allergy; White ash allergy; White bean llergy; White hickory allergy; White pine allergy; White-faced hornet enom allergy; Whitefish (Inconnu) allergy; Wild rye grass allergy; Willow allergy; Wormwood allergy; Yeast allergy; Yellow dock llergy; Yellow hornet venom allergy;
Endocrine, nutritional and etabolic diseases: Diabetes mellitus; Thyroid diseases
Diseases of the circulatory system: Acute myocardial infarction
Diseases of the respiratory system: Chronic obstructive pulmonary disease
Diseases of the digestive system: Primary biliary cholangitis; Inflammatory bowel disease;
Coeliac disease; Autoimmune hepatitis; Diseases of liver
Diseases of the musculoskeletal system or connective tissue: Rheumatoid arthritis
Development anomalies: Chromosomal anomalies
Other: Goodpasture syndrome.

### LIST OF REFERENCE SIGNS

FIG. 1a, 1b
100 Labeling nanostructure
101 Analyte
102 Substrate
103 Polynucleotide-based nanostructure
104 First binding sites
104a First binding site sequences, which peeks out of nanostructure
104b First binding site reagent
105 Second binding sites
105a Second binding site sequence, which peeks out of nanostructure/(anchor strand)
105b Second binding site reagent (biotin or streptavidin)
106 Functionalized substituent
106b binding reagent of substituent
107 Detectable label
107a Nanoparticle
108 Target
108a Target binding site
108b Target molecule
109 Third binding site
109a Third binding site, which peaks out of nanostructure
109b Third binding site reagent
110 Control
110a Control binding site
110b Control molecule
FIG. 2a, 2b, 2c
211 Test device
212 Test substrate
213 Region containing the functionalized nanoparticles plus its mobile conjugate
214 Fluid sample
215 Test lines with immobilized antibodies for letting the conjugate bind to the antibodies
216 Method of producing the test device
217, 218 method steps of method 216
FIGS. 3a, 3b, 3c
300 Labeling nanostructure
301 Analyte
302 Paper-based lateral flow substrate
303 Polynucleotide-based nanostructure
306 Detectable label
306b Functionalized substituent
305 Second binding site
305a Second binding site sequence, which peeks out of nanostructure
309a Third binding site, which peaks out of nanostructure
309b Third binding site reagent
311 Waste pad
312 Absorption pad
308a Binding zone
315 Test line
314 Drop of sample solution
318 biotin-DNA/DNA origami mixture
330 Control
FIG. 4
402 Substrate
415 T-line
420 C-line
FIG. 5
515a Test line for LFA using 40 nm gold particles.
515b Test line for LFA using detectable nanostructures.
FIG. 6
600 Labeling nanostructure
601 Analyte
602 Substrate
603 Polynucleotide-based nanostructure
604 Antibody
605 Immobilized antibody
606 Detectable label
607 Test line
608 TEM image of labeling nanostructure
FIG. 7
700 Labeling nanostructure
701 bead
702 antibody
703 analyte
704 detection antibody
705 nanostructure
706 detectable label
801: polynucleotide scaffold
802: polynucleotide staples
803: polynucleotide nanostructure with predefined size and shape (e.g. elongated filament with 428 nm length and 8 nm diameter)
804: polynucleotide-modified detection antibody with binding capability to the first binding site of the polynucleotide nanostructure (e.g. monoclonal anti-troponin I antibody 1)
805: polynucleotide nanostructure modified with detection antibody
806: polynucleotide-modified label with binding capability to the second binding site of the polynucleotide nanostructure
807: fully assembled labeling nanostructure bound with detection antibody to the first binding site and labels to the second binding sites
808: application of the fully assembled labeling nanostructure in a microfluidic sandwich immunoassay
809: analyte (e.g. cardiac troponin I-C complex)
810: capture antibody (e.g. monoclonal anti-troponin I antibody 2)
811: substrate (e.g. nitrocellulose, well-plate, microfluidic channel, microbead)
901: result of agarose gel electrophoresis (1% agarose)
902: molecular ruler
903: polynucleotide scaffold
904: polynucleotide nanostructure with precise, pre-defined size and molecular weight and excess staples
905: transmission electron micrograph of the elongated filament polynucleotide nanostructure
1001: detection of cardiac troponin I with a colloidal gold based lateral flow assay without signal amplification
1002: the target band is not detectable at low concentration
1003: result of signal amplification on a troponin I colloidal gold based lateral flow assay
1004: the target band is detectable at low concentration
1101: integration of the polynucleotide nanostructure in a sequential immunoassay protocol on the surface of a well (microfluidic wells or 96-well/384-well plate). Signal is read out with an UV-VIS spectrometer, in this case a plate reader (fluorescence or absorption depending on the label).
1201: UV-VIS readout at 125 picomolar concentration of the analyte (cardiac troponin I-C complex)
1202: signal amplification
1203: detection with single colloidal gold labels without signal amplification
1301: UV-VIS readout of nanoparticle coupling effects due to polynucleotide nanostructure mediated assembly in close proximity
1302: spectral shift of 80 nm due to nanoparticle coupling effects on the labeling nanostructure, no shift is observed for single nanoparticles
1401: labeling nanostructure configured with a predefined number of one colloidal gold label used in a lateral flow assay
1402: labeling nanostructure configured with a predefined number of three colloidal gold labels used in a lateral flow assay
1403: labeling nanostructure configured with a predefined number of eighteen colloidal gold labels used in a lateral flow assay

1. A labeling nanostructure usable in immunoassays, comprising
a polynucleotide-based nanostructure having
one or more first binding sites for binding the polynucleotide-based nanostructure to an analyte, and
a predefined number N>1 of second binding sites, the number being defined by a predefined sequence of nucleotides, wherein each second binding site is configured to bind a detectable label.

2. The labeling nanostructure of claim 1, wherein the polynucleotide-based nanostructure has a predefined size and/or a predefined shape.

3. The labeling nanostructure of claim 1, wherein the polynucleotide-based nanostructure comprises one or more scaffold strands.

4. The labeling nanostructure of claim 1, wherein the predefined number N of second binding sites are configured to bind a label suitable for being detected in an immunoassay, the detectable label being selected from the group of detectable labels comprising an enzyme or fluorescent label, or a particle label.

5. The labeling nanostructure of claim 1, wherein the predefined number N is 1<N<1000, 10<=N<1000, 10<=N<750, 10<=N<500, 10<=N<400, 10<=N<350, or 10<=N<300.

6. The labeling nanostructure of claim 1, wherein each second binding site is adapted for binding a detectable label, whose detection produces a first signal having a predefined first value V1, and wherein the predefined number N of second binding sites provide a sum of N detectable labels, whose detection produces a second signal having a predefined second value V2, which is a known amplification Amp of the first value, i.e. V2=Amp*V1.

7. The labeling nanostructure of claim 1, which comprises a number N of detectable labels, in each case one detectable label being bound to each of the predefined number N of second binding sites.

8. A test device for running an immunoassay, the test device comprising,
a test substrate comprising a plurality of labeling nanostructures according to claim 1.

9. The test device according to claim 8, wherein the plurality of labeling nanostructures comprises groups of labeling nanostructures, wherein the groups distinguish by their label sizes, wherein within each group a uniform label size prevails.

10. The test device according to claim 8, being adapted for running a lateral flow immunoassay, the test substrate comprising a strip including a porous membrane, which contains the plurality of labeling nanostructures, which comprise the polynucleotide-based nanostructures with their first binding sites having a first binding site reagent, which is capable to bind to an analyte to be detected by the immunoassay.

11. The test device according to claim 10, the test substrate comprising a binding area, where target reagents are bound to the test substrate and thereby immobilized and a solution containing the analyte being labeled by the labeling nanostructures is capable to flow along the test substrate to the binding area, which is configured to bind the analyte, which is labeled by the labeling nanostructures to the immobilized target reagents.

12. A method of producing the labeling nanostructure according to claim 1, the method comprising the step:
synthesizing a polynucleotide-based nanostructure based on a predefined sequence of nucleotides, including one or more first binding sites for binding the polynucleotide-based nanostructure to an analyte, and including a predefined number N>1 of second binding sites being defined by the predefined sequence of nucleotides, each second binding site being configured for binding a detectable label.

13. A method of producing the test device according to claim 8, the method comprising the steps of
providing a test substrate;
applying to the test substrate a plurality of labeling nanostructures.

14. A method for providing signal amplification to an immunoassay method, which uses a single detectable label, the method for providing signal amplification provides the following steps:
providing the labeling nanostructure according to claim 1 for acting as an amplifier;
replacing and/or adding, in the immunoassay method, the single detectable label by the labeling nanostructure, which comprises a number N>1 of detectable labels.

15. Use of the labeling nanostructure according to claim 1 for acting as a signal amplified label in an immunoassay, e.g., in a lateral flow immunoassay (LFA), or in a bead-based immunoassay method.

16. The labeling nanostructure of claim 3, wherein the polynucleotide-based nanostructure comprises a DNA origami.

17. The labeling nanostructure of claim 4, wherein the detectable label comprises a visible or a non-visible particle label, e.g. a fluorescent particle, a liposome, a gold particle, a latex particle, a Q-Dot, a carbon nanotube, a silver particle, a silver coated particle, or a cellulose particle.

18. The test device of claim 8, for running a lateral flow immunoassay.

19. The test device of claim 8, wherein the test substrate comprises a porous substrate.
